SEQUENCE LISTING (12 sequences)

SEQ ID NO: 1 - protein, 127 aa, homo sapiens
MSFSGKYQLQSQENFEAFMKAIGLPEELIQKGKDIKGVSEIVQNGKHFKFTITAGSKVIQNEFTVGEECELETMTGEKVKTVVQLEGDNKLVTTFKNIKSVTELNGDIITNTMTLGDIVFKRISKRI

SEQ ID NO: 2 - protein, 132 aa, Homo sapiens
MAFDSTWKVDRSENYDKFMEKMGVNIVKRKLAAHDNLKLTITQEGNKFTVKESSAFRNIEVVFELGVTFNYNLADGTELRGTWSLEGNKLIGKFKRTDNGNELNTVREIIGDELVQTYVYEGVEAKRIFKKD

SEQ ID NO: 3 - protein, 133 aa, homo sapiens [sequence omitted]

SEQ ID NO: 4 - protein, 132 aa, homo sapiens
MCDAFVGTWKLVSSENFDDYMKEVGVGFATRKVAGMAKPNMIISVNGDVITIKSESTFKNTEISFILGQEFDEVTADDRKVKSTITLDGGVLVHVQKWDGKSTTIKRKREDDKLVVECVMKGVTSTRVYERA

SEQ ID NO: 5 - protein, 135 aa, homo sapiens
MATVQQLEGRWRLVDSKGFDEYMKELGVGIALRKMGAMAKPDCIITCDGKNLTIKTESTLKTTQFSCTLGEKFEETTADGRKTQTVCNFTDGALVQHQEWDGKESTITRKLKDGKLVVECVMNNVTCTRIYEKVE

SEQ ID NO: 6 - protein, 128 aa, homo sapiens
MAFTGKFEMESEKNYDEFMKLLGISSDVIEKAHNFKIVTEVQQDGQDFTWSQHYYGGHTMTNKFTVGKESNIQTMGGKTFKATVQMEGGKLVVNFPNYHQTSEIVGDKLVEVSTIGGVTYERVSKRLA

SEQ ID NO: 7 - protein, 132 aa, homo sapiens [sequence omitted]

SEQ ID NO: 8 - protein, 132 aa, homo sapiens
MSNKFLGTWKLVSSENFDDYMKALGVGLATRKLGNLAKPTVIISKKGDIITIRTESTFKNTEISFKLGQEFEETTADNRKTKSIVTLQRGSLNQVQRWDGKETTIKRKLVNGKMVAECKMKGVVCTRIYEKV

SEQ ID NO: 9 - protein, 132 aa, homo sapiens
MVEPFLGTWKLVSSENFEDYMKELGVNFAARNMAGLVKPTVTISVDGKMMTIRTESSFQDTKISFKLGEEFDETTADNRKVKSTITLENGSMIHVQKWLGKETTIKRKIVDEKMVVECKMNNIVSTRIYEKV

SEQ ID NO: 10 - protein, 126 aa, danio rerio
MAFSGTWQVYAQENYEEFLRAISLPEEVIKLAKDVKPVTEIQQNGSDFTITSKTPGKTVTNSFTIGKEAEITTMDGKKLKCIVKLDGGKLVCRTDRFSHIQEIKAGEMVETLTVGGTTMIRKSKKI

SEQ ID NO: 11 - protein, 134 aa, danio rerio
MVDKFVGTWKMTTSDNFDEYMKAIGVGFATRQVGNRTKPNLVVCVDEQGLICMKSQSTFKTTEIKFKLNEPFEETTADDRKTTTVMTIENGKLVQKQTWDGKESTIEREVSDGKLIAKCKMGDVVAVRTYVKEA

SEQ ID NO: 12 - protein, 140 aa, homo sapiens
MIDQLQGTWKSISCENSEDYMKELGIGRASRKLGRLAKPTVTISTDGDVITIKTKSIFKNNEISFKLGEEFEEITPGGHKTKSKVTLDKESLIQVQDWDGKETTITRKLVDGKMVVESTVNSVICTRTYEKVSSNSVSNS

(19) United States
(12) Patent Application Publication
(10) Pub. No.: US 2015/0285812 A1
(43) Pub. Date: Oct. 8, 2015

(54) Fluorescence-Labelled Fatty Acids and Uses Thereof

(71) Applicant: SANOFI, Paris (FR)

(72) Inventors: Stefan Petry, Frankfurt am Main (DE); Marc Nazare, Frankfurt am Main (DE); Thorsten Schmidt, Frankfurt am Main (DE); Hans Matter, Frankfurt am Main (DE)

(21) Appl. No.: 14/426,044

(22) PCT Filed: Sep. 4, 2013
(86) PCT No.: PCT/EP2013/068275

§ 371 Date: Mar. 4, 2015

(30) Foreign Application Priority Data
Sep. 6, 2012 (EP) 12306069.1

(51) Int. Cl. G01N 33/58

(52) U.S. Cl. CPC G01N 33/582

(57) ABSTRACT

The present invention relates to a composition comprising (i) a fluorescent-labelled fatty acid and (ii) a fatty acid binding compound, wherein (a) the fatty acid component of the fluorescent-labelled fatty acid binds the fatty acid binding compound and (b) the fluorescent component of the fluorescent-labelled fatty acid and the fatty acid binding compound interact to elicit FRET (Förster resonance energy transfer) effects. Moreover, the invention is directed to a method for identifying and/or characterizing a compound of interest by contacting a fluorescent-labelled fatty acid with a fatty acid binding compound under conditions that allow for binding and for FRET (Förster resonance energy transfer) effects, and then contacting the fluorescent-labelled fatty acid bound to the fatty acid binding compound with a compound of interest and determining the change in fluorescence. In addition, the invention pertains to corresponding kits of parts and uses of the compositions and methods.

### FIELD OF THE INVENTION

The present invention relates to a composition comprising (i) a fluorescent-labelled fatty acid and (ii) a fatty acid binding compound, wherein (a) the fatty acid component of the fluorescent-labelled fatty acid binds the fatty acid binding compound and (b) the fluorescent component of the fluorescent-labelled fatty acid and the fatty acid binding compound interact to elicit FRET (Förster resonance energy transfer) effects. Moreover, the invention is directed to a method for identifying and/or characterizing a compound of interest by contacting a fluorescent-labelled fatty acid with a fatty acid binding compound under conditions that allow for binding and for FRET (Förster resonance energy transfer) effects, and then contacting the fluorescent-labelled fatty acid bound to the fatty acid binding compound with a compound of interest and determining the change in fluorescence. In addition, the invention pertains to corresponding kits of parts and uses of the compositions and methods of the invention.

### BACKGROUND OF THE INVENTION

In biology the interactions between physiological components such as proteins, fats, sugars, nucleotides, etc. regulate all aspects of life. In particular, proteins can serve as enzymes, structuring agents in cells and tissue, as gene regulatory elements, physiological messengers as well as in defense, transport and storage.

The protein albumin is the most frequent transport protein in blood plasma and tissue fluids. It binds many endogenous and exogenous substances such as fatty acids, vitamins, hormones, drugs, toxins and waste products such as heme and bilirubin and thereby directly and indirectly affects many body functions. Physiologically, albumin's most prominent role is the binding and transport of fatty acids, which are related to various metabolic and cardiovascular diseases. Serum albumin is of particular relevance for drugs due to its binding affinity to a multitude of drug substances and, thereby, influences the free and active plasma concentration of the drugs. For characterizing new medically active candidates, their transport and binding properties in plasma is an important aspect.

The ligand binding of human serum albumin (HSA) has been investigated thoroughly, for example by ultrafiltration methods, electrophoresis, equilibrium dialysis, X-ray diffraction (crystal structures) as well as spectrofluorometric processes. Especially crystal structure studies of albumin with and without ligands gave insights into albumin's binding characteristics. However, rigid crystal structures are artifacts and hardly reflect physiological conformations and properties of the dynamic albumin protein in solution or in blood plasma. Co-crystallization of proteins is usually performed under ligand concentrations, which are dramatically higher than the physiological concentrations. Under these artificial conditions, binding sites might be identified, which have no physiological relevance. In addition the relative affinities of binding sites can not be determined. Spectroscopic methods such as absorption and fluorescence, which can be performed in aqueous media under physiological conditions, retain the conformational flexibility of albumin, are good alternative for investigating the binding sites of serum albumin.

Fluorescent dyes are frequently used for spectroscopically marking cell constituents, proteins and other physiological compounds. Derivatives of 4-nitrobenzo-2-oxa-1,3-diazole (NBD) and 5-(dimethylamino)naphthalene-1-sulfonyl (dansyl) are typical examples of fluorescent marker dyes.

Presently, there are few reports relating to the investigation of albumin binding using dye labels under physiological conditions.

Rohacova et al. (J. Phys. Chem. B., 2010, 114, 4710-4716) investigated the binding of human serum albumin to bile acids by means of fluorescent cholic acid derivatives. The binding of bile acids to plasma albumin critically determines the bile acid plasma level and is an indicator of liver function. In particular, the authors used fluorescent cholic acid derivatives for assessing the role of bile acid-human serum albumin-complexes in hepatic uptake.

In 2011 Rohacova et al. (J. Phys. Chem. B., 2011, 115, 10518-10524) further investigated the binding of human serum albumin to bile acids using four dansyl (Dns) derivatives of cholic acid. Using both steady-state and time-resolved fluorescence, formation of Dns-ChA-HSA complexes was confirmed for two binding sites, the corresponding binding constants were determined and their distribution between bulk solution and HSA microenvironment was estimated.

It is the object of the present invention to provide compositions, kits and methods for identifying and/or characterizing compounds of interest with respect to their binding affinity to other compounds of biological relevance. In particular, it is an object of the present invention to identify and/or characterize compounds of interest that bind to fatty acid binding compounds such as albumins and fatty acid binding proteins (FABP).

These and further objectives are solved by the aspects of present invention such as a composition comprising (i) a fluorescent-labelled fatty acid and (ii) a fatty acid binding compound, wherein (a) the fatty acid component of the fluorescent-labelled fatty acid binds the fatty acid binding compound and (b) the fluorescent component of the fluorescent-labelled fatty acid and the fatty acid binding compound interact to elicit FRET (Förster resonance energy transfer) effects.

Surprisingly, it was found that a fluorescent-labelled fatty acid and a fatty acid binding compound, which interact to elicit FRET interactions, have utility for identifying and characterizing a compound of interest. If the compound of interest binds to the same binding site as the fluorescent-labelled fatty acid on the fatty acid binding compound, the compound of interest competitively displaces the fluorescent-labelled fatty acid, thereby abrogates FRET interactions, and thus decreases fluorescence.

In other words, the composition of the present invention is a sensor essentially comprising a binary binding and signalling system. The signal trigger of this binary system is the FRET effect or the loss thereof if a competitor displaces the signalling fluorescent-labelled fatty acid from the fatty acid binding compound.

### ASPECTS OF THE INVENTION

In a first aspect, present invention relates to a composition comprising

(i) a fluorescent-labelled fatty acid and
(ii) a fatty acid binding compound,
wherein
(a) the fatty acid component of the fluorescent-labelled fatty acid binds the fatty acid binding compound and
(b) the fluorescent component of the fluorescent-labelled fatty acid and the fatty acid binding compound interact to elicit FRET (Förster resonance energy transfer) effects.

In a second aspect, present invention relates to a method for identifying and/or characterizing a compound of interest, comprising the steps of:

(i) providing a fluorescent-labelled fatty acid,
(ii) providing a fatty acid binding compound that binds to the fatty acid and interacts with the fatty acid to elicit FRET (Förster resonance energy transfer) effects,
(iii) contacting the fluorescent-labelled fatty acid with the fatty acid binding compound under conditions that allow for (a) the binding of the fatty acid to the fatty acid binding compound and for (b) FRET (Förster resonance energy transfer) effects,
(iv) contacting the fluorescent-labelled fatty acid bound to the fatty acid binding compound with a compound of interest under conditions that allow for (a) the binding of the fatty acid to the fatty acid binding compound and for (b) FRET (Förster resonance energy transfer) effects,
(v) determining the change in fluorescence, and
(vi) optionally calculating the binding affinity of the compound of interest to the fatty acid binding compound.

In a third aspect, present invention relates to a method for identifying and/or characterizing a compound of interest comprising a method according to the second aspect, wherein said method is repeated for the same compound of interest at least once with variation in at least one of the following parameters:
(a) the fluorescent-labelled fatty acid of step (i),
(b) the fatty acid binding compound of step (ii) and/or
(c) the conditions of steps (iii) and/or (iv).

In a fourth aspect, present invention relates to a kit of parts comprising at least

(i) a fluorescent-labelled fatty acid and
(ii) a fatty acid binding compound,
wherein
(a) the fatty acid component of the fluorescent-labelled fatty acid binds the fatty acid binding compound and
(b) the fluorescent component of the fluorescent-labelled fatty acid and the fatty acid binding compound interact to elicit FRET (Förster resonance energy transfer) effects.

In a fifth aspect, present invention relates to the use of a composition and/or a kit of parts according to one of the aspects of present invention for performing a method according of present invention.

In a sixth aspect, present invention relates to a method of manufacturing a composition of present invention comprising admixing a fluorescent-labelled fatty acid and a fatty acid binding compound under conditions allowing for a binding of the fatty acid component of the fluorescent-labelled fatty acid and the fatty acid binding compound.

In a seventh aspect, present invention relates to a method of manufacturing a kit of present invention comprising assembling the different components of the kit to form a spatial and/or functional unit. In one embodiment, the method further comprises packaging the different components into one or more containers.

The different aspects of present invention will now be described in more detail in the detailed description of present invention:

The fluorescent component for use in the different aspects of present invention, such as in the fluorescent-labelled fatty acid is any fluorescent component suitable for FRET interactions, e.g. FRET interactions with a tryptophan in a polypeptide. In one embodiment, the fluorescent component is selected from the group consisting of 4-nitrobenzo-2-oxa-1,3-diazole (NBD), 5-(dimethylamino)naphthalene-1-sulfonyl (dansyl), Dansyl Rhodamin, Alizarin, Texas Red Marine Blue, Green Fluorescent Protein, Flourescamine, and Flourescein. In a particular embodiment, the fluorescent compound is 4-nitrobenzo-2-oxa-1,3-diazole (NBD).

The fatty acid component for use in the fluorescent-labelled fatty acid can be any fatty acid capable of binding to a fatty acid binding compound. In one embodiment, the fatty acid component is selected from the group consisting of linear or branched, saturated or unsaturated fatty acids. In one embodiment, the fatty acid component is selected from the group consisting of linear and saturated fatty acids. In a particular embodiment, the fatty acid component for use in the invention is selected from the group consisting of linear and saturated fatty acids having 2 to 25 carbon atoms. In another embodiment, the fatty acid component for use in the invention is selected from the group consisting of linear and saturated fatty acids having 8 to 20 carbon atoms. In another embodiment, the fatty acid component for use in the invention is selected from the group consisting of linear and saturated fatty acids having 10 to 18 carbon atoms. In a particular embodiment, the fatty acid component for use in the invention is selected from the group consisting of linear and saturated fatty acids having 10 to 16 carbon atoms.

The fatty acid component of the different aspects of present invention can be a saturated or unsaturated, a linear or branched fatty acid. Examples of fatty acid components comprise e.g. ethanoic, propanoic, butanoic, pentanoic, hexanoic, heptanoic, octanoic, nonanoic, decanoic, undecanoic, dodecanoic, tridecanoic, butadecanoic, pentadecanoic, hexadecanoic acid, heptadecanoic, octadecanoic acid, etc. or a mono-, di- or triacylglyceride. In one embodiment, fatty acid components of the different aspects of present invention concern linear fatty acids. In another embodiment, fatty acid components of the different aspects of present invention concern unsaturated fatty acids. Particular embodiments of the different aspects of present invention concern linear and saturated fatty acids. Specific and particular embodiments of the fatty acid component are linear and saturated ethanoic, propanoic, butanoic, pentanoic, hexanoic, heptanoic, octanoic, nonanoic, decanoic, undecanoic, dodecanoic, tridecanoic, butadecanoic, pentadecanoic, hexadecanoic acid, heptadecanoic, octadecanoic acids, etc.

Further particular examples of the fatty acid component are mono-, di- or triglycerides. In this respect, the inventors have found that mono- di- or triacylglycerides competitively bind to the high affinity fatty acid binding site. This finding allows for the first time to use mono-, di- or triglycerides e.g. the fluorescence-labelled monoacylglycerols as described in S. Petry et al., (J. Lipid Res. 46 (2005) 603) as attractive substrates for practicing the present invention. Examples of mono- di- or triglycerides for practicing the different aspects of present invention are monoerucin, monolaurin, monomyristin, monopalmitin, dipalmitelaidin, 1,3-dipalmitolein, 1-palmitin-3-olein, 1,2-dioleoyl glycerol, 1,3-diarachidonin, monostearin, tripalmitolein, 1,3-dioleoyl-2-palmitoyl-glycerol, hexadecanoic acid 3-hexadecanoyloxy-2-hydroxypropyl ester, 1,2-dipalmitolyl-sn-glycerol, 1,3-dipalmitin, 1,3-distearyl glycero, trielaidin, tripetroselaidin, trilinolein, trimyristin, tripalmitin, glyceroltristearate, triarachidin. In a particular embodiment, the mono- di- or triglycerides for practicing the different aspects of present invention are caproic acid (C₆), caprylic acid (C₈), capric acid (C₁₀), lauric acid (C₁₂), myristic acid (C₁₄) and palmitic acid (C₁₆) stearic acid (C18).

The fatty acid component for use in the different aspects of present invention can be labelled or unlabelled. According to one aspect of present invention, the fatty acid component is labelled. In a particular embodiment, the fatty acid component is labelled with a fluorescence marker as herein described.

The fatty acid binding compound for use in the different aspects of present invention is any compound or particle capable of binding to at least one fatty acid. In one embodiment, the fatty acid binding compound for use in the different aspects of present invention is a linear or branched, saturated or unsaturated fatty acid. In one embodiment, the fatty acid binding compound for use in the different aspects of present invention is a linear and saturated fatty acid as described above. In one embodiment, the particle capable of binding to at least one fatty acid is a particle comprising a compound capable of binding to at least one fatty acid. In particular embodiments the fatty acid binding compound is selected from the group consisting of proteins comprising at least one tryptophan. In one embodiment, the fatty acid binding compound is selected from the group consisting of albumin such as albumin of any species, HDL, LDL, VLDL, and fatty acid binding proteins (FABPs). In one embodiment said albumin is human serum albumin.

Fatty acid binding proteins (FABPs) are carrier proteins for fatty acids and other lipophilic substances such as eicosanoids and retinoids. In eukaryotes, a family of FABPs is known to exist, comprising the following members: FABP 1-12 and FABP5-like proteins 1-7. The FABPs are conceived to transport lipophilic molecules from the outer cell membrane to intracellular receptors such as PPARs. In the context of the present application, any protein able to carry fatty acids and other lipophilic substances such as eicosanoids and retinoids from the outer cell membrane to intracellular FABP receptors is considered as FABP in the ambit of present application. In one embodiment, the FABPs of present invention are FABP 1-12 (i.e. any of FABP1, FABP2, FABP3, FABP4, FABP5, FABP6, FABP7, FABP8, FABP9, FABP10, FABP11, FABP12) and FABP5-like proteins 1-7 (i.e. any of FABP5-like protein 1, 2, 3, 4, 5, 6 or 7).

The sequences of the above-identified proteins and their encoding nucleic acids can be retrieved under the above accession numbers e.g. at the NCBI: NCBI is the national centre for biotechnology information (postal address: National Centre for Biotechnology Information, National Library of Medicine, Building 38A, Bethesda, Md. 20894, USA; web-address: http://www.ncbi.nlm.nih.gov).

The GenBank accession numbers of FABP1 to FABP12 are as follows:

FABP1 according to GenBank entry CAG46887 and, in a particular embodiment, CAG46887.1 (hs, 127 aa, SEQ ID NO:1), FABP2 according to GenBank entry AAH69617 and, in a particular embodiment, AAH69617.1 (132 aa, hs, SEQ ID NO:2), FABP3 according to GenBank entry CAG33148 and, in a particular embodiment, CAG33148.1 (SEQ ID NO:3, hs, 133 aa), FABP4 according to GenBank entry CAG33184 and, in a particular embodiment, CAG33184.1 (SEQ ID NO:4, hs, 132 aa), FABP5 according to GenBank entry AAH70303 and, in a particular embodiment, AAH70303.1 (SEQ ID NO:5, hs, 135 aa), FABP6 according to GenBank entry CAB65728 and, in a particular embodiment, CAB65728.1 (SEQ ID NO:6, hs, 128 aa), FABP7 according to GenBank entry CAG33338 and, in a particular embodiment, CAB33338.1 (SEQ ID NO:7, hs, 132 aa), FABP8 (myelin P2 protein) according to GenBank entry AAH34997 and, in a particular embodiment, AAH34997.1 (SEQ ID NO:8, hs, 132 aa), FABP9 according to GenBank entry NP_{—}001073995 and, in a particular embodiment, NP_{—}001073995.1 (SEQ ID NO:9, homo sapiens, 132 aa), FABP10 according to GenBank entry AAI64928 and, in a particular embodiment, AAI64928.1 (Danio rerio, zebrafish, 126 aa, SEQ ID NO:10), FABP11 (11a) according to GenBank entry NP_{—}001004682 and, in a particular embodiment, NP_{—}001004682.1 (Danio rerio, zebrafish, 134 aa, SEQ ID NO:11), FABP12 according to GenBank entry NP-001098751 and, in a particular embodiment, NP_{—}001098751.1 (SEQ ID NO:12, homo sapiens, 140 aa).

The sequences of FABPs 1-12 according to the above accession numbers are disclosed in the attached sequence listing that, with its whole content and disclosure, is a part of this specification.

A compound in the context of the different aspects of present invention can be any biological substance (e.g. protein, polypeptide, nucleic acid, lipid, carbohydrate or combination thereof) or chemical substance or natural product extract, either purified, partially purified, synthesized or manufactured by means of biochemical or molecular biological methods.

The compound of interest in the context of the different aspects of present invention can be any such compound that binds to one or more fatty acid binding compounds, e.g. to those fatty acid binding compounds as exemplified above such as one or more albumins. Example compounds comprise Ibuprofen, ketoprofen, warfarin, lipids, fatty acids, cholesterol, palmitic acid, myristic acid and others known in the art

In the examples, the general inventive concept is illustrated for HSA, NBD and linear unsaturated fatty acids having 8 to 16 carbon atoms. HSA is of particular relevance for drug screening because it strongly influences drug availability in the blood serum, as previously noted. However, it is understood that the invention is more broadly directed to any system of fluorescent-labelled fatty acid and fatty acid-binding compound.

In a particular embodiment, the composition of the invention is one, wherein
(i) the fluorescent component is 4-nitrobenzo-2-oxa-1,3-diazole (NBD),
(ii) the fatty acid component is selected from the group consisting of linear and saturated (or unsaturated) fatty acids having 8 to 25 carbon atoms, and
(iii) the fatty acid binding compound is albumin.

In one embodiment, the fatty acid component is selected from the group consisting of linear and saturated (or unsaturated) fatty acids having 8 to 20 carbon atoms. In another embodiment, the fatty acid component is selected from the group consisting of linear and saturated (or unsaturated) fatty acids having 10 to 18 carbon atoms. In a particular embodiment, the fatty acid component is selected from the group consisting of linear and saturated (or unsaturated) fatty acids having 12 to 16 carbon atoms.

In one embodiment, the fatty acid binding compound is human serum albumin.

In a further particular embodiment of the composition comprising albumin the concentration of the fluorescent-labelled fatty acid is about 2 μM to about 50 μM, or 2 μM to 50 μM. In one embodiment, the concentration of the fluorescent-labelled fatty acid is about 10 μM to about 40 μM, or 10 μM to 40 μM. In another embodiment, the concentration of the fluorescent-labelled fatty acid is about 10 μM to about 35 μM, or 10 μM to 35 μM. In another embodiment, the concentration of the fluorescent-labelled fatty acid is about 10 μM to about 30 μM, or 10 μM to 30 μM. In yet another embodiment, the concentration of the fluorescent-labelled fatty acid is about 15 μM to about 25 μM, or 15 μM to 25 μM, or about 25 μM.

For other combinations of fatty acid and fatty acid binding compound, the fluorescent component for the fatty acid, the fatty acid type, the optimum concentrations thereof and the binding conditions can be determined by routine techniques and without any undue burden in view of the present disclosure.

In a further aspect, the present invention is directed to a method for identifying and/or characterizing a compound of interest, comprising the steps of:
(i) providing a fluorescent-labelled fatty acid,
(ii) providing a fatty acid binding compound that binds to the fatty acid and interacts with the fatty acid to elicit FRET (Förster resonance energy transfer) effects,
(iii) contacting the fluorescent-labelled fatty acid with the fatty acid binding compound under conditions that allow for (a) the binding of the fatty acid to the fatty acid binding compound and for (b) FRET (Förster resonance energy transfer) effects,
(iv) contacting the fluorescent-labelled fatty acid bound to the fatty acid binding compound with a compound of interest under conditions that allow for (a) the binding of the fatty acid to the fatty acid binding compound and for (b) FRET (Förster resonance energy transfer) effects,
(v) determining the change in fluorescence, and
(vi) optionally calculating the binding affinity of the compound of interest to the fatty acid binding compound.

The above method is simple and reliable because the fluorescence signal in step (v) depends on fatty acid binding to and FRET interactions with the fatty acid binding compound. A loss of fluorescence intensity is the result of the competitive binding of the compound of interest to the fatty acid binding compound and the subsequent release of the fluorescent-labelled fatty acid, which can be evaluated without further sample treatment, i.e. no modification, purification or characterizing method such as chromatography is required for identifying and/or characterizing the fatty acid or the compound of interest.

In one embodiment, the fluorescent component for practicing the method of the invention is selected from the group consisting of 4-nitrobenzo-2-oxa-1,3-diazole (NBD) and 5-(dimethyl-amino)naphthalene-1-sulfonyl (dansyl). In a particular embodiment, the fluorescent compound is 4-nitrobenzo-2-oxa-1,3-diazole (NBD).

In one embodiment, the fatty acid component for practicing the method of the invention is selected from the group consisting of linear or branched, saturated or unsaturated fatty acids. In a particular embodiment, the fatty acid component is selected from the group consisting of linear and saturated fatty acids. In one embodiment, the fatty acid component of the fluorescent-labelled fatty acid is selected from the group consisting of linear and saturated fatty acids having 2 to 25 carbon atoms. In one embodiment, the fatty acid component is selected from the group consisting of linear and saturated fatty acids having 8 to 20 carbon atoms. In another embodiment, the fatty acid component is selected from the group consisting of linear and saturated fatty acids having 10 to 18 carbon atoms. In a particular embodiment, the fatty acid component is selected from the group consisting of linear and saturated fatty acids having 10 to 16 carbon atoms.

In one embodiment, the fatty acid binding compound for practicing the method of the invention is selected from the group consisting of proteins comprising at least one tryptophan. In one embodiment, the fatty acid binding compound is selected from the group consisting of albumin, HDL, LDL, VLDL. In one embodiment, said albumin is human serum albumin.

The method of the present invention is particularly useful for assessing the binding of drugs to albumin in the context of preclinical and clinical studies. Therefore, in a particular embodiment the method of the invention is one, wherein
(i) the fluorescent component is 4-nitrobenzo-2-oxa-1,3-diazole (NBD),
(ii) the fatty acid component is selected from the group consisting of linear and saturated fatty acids having 2 to 25,
(iii) the fatty acid binding compound is albumin.

In one embodiment, the fatty acid component is selected from the group consisting of linear and saturated fatty acids having 8 to 20 carbon atoms. In another embodiment, the fatty acid component is selected from the group consisting of linear and saturated fatty acids having 10 to 18 carbon atoms. In a particular embodiment, the fatty acid component is selected from the group consisting of linear and saturated fatty acids having 10 to 16 carbon atoms.

In one embodiment, the fatty acid binding compound is human serum albumin.

In one embodiment of the method of the present invention, the concentration of the fluorescent-labelled fatty acid is about 2 μM to about 50 μM, or 2 μM to 50 μM. In another embodiment, the concentration of the fluorescent-labelled fatty acid is about 5 μM to about 40 μM, or 5 μM to 40 μM. In another embodiment, the concentration of the fluorescent-labelled fatty acid is about 5 μM to about 30 μM, or 5 μM to 30 μM. In another embodiment, the concentration of the fluorescent-labelled fatty acid is about 10 μM to about 30 μM, or 10 μM to 30 μM. In yet another embodiment, the concentration of the fluorescent-labelled fatty acid is about 12 μM to about 25 μM, or 12 μM to 25 μM, or about 25 μM.

In an optional step, the binding affinity of the compound of interest to the fatty acid binding compound can be calculated. In a particular embodiment, the binding affinity is defined as the concentration of the fatty acid binding compound of interest corresponding to the concentration of the fluorescent-labelled fatty acid at the half maximum fluorescent signal.

The conditions and parameters for practicing the method of the invention can be determined by the average skilled person without undue burden and without inventive skill in view of the common general knowledge in the relevant field and the examples presented below, which are exemplified for albumin. In particular, conditions that allow for (a) the binding of the fatty acid to the fatty acid binding compound and for (b) FRET (Förster resonance energy transfer) effects are presented in the examples.

In a further aspect the present invention is directed to a method for identifying and/or characterizing a compound of interest, comprising the steps of:
(i) providing a fluorescent-labelled fatty acid,
(ii) providing a fatty acid binding compound that binds to the fatty acid and interacts with the fatty acid to elicit FRET (Förster resonance energy transfer) effects,
(iii) contacting the fluorescent-labelled fatty acid with the fatty acid binding compound under conditions that allow for (a) the binding of the fatty acid to the fatty acid binding compound and for (b) FRET (Förster resonance energy transfer) effects,
(iv) contacting the fluorescent-labelled fatty acid bound to the fatty acid binding compound with a compound of interest under conditions that allow for (a) the binding of the fatty acid to the fatty acid binding compound and for (b) FRET (Förster resonance energy transfer) effects,
(v) determining the change in fluorescence, and
(vi) optionally calculating the binding affinity of the compound of interest to the fatty acid binding compound,
   wherein said method is repeated for the same compound of interest at least once with variation in at least one of the following parameters:
(d) the fluorescent-labelled fatty acid of step (i),
(e) the fatty acid binding compound of step (ii) and/or
(f) the conditions of steps (iii) and/or (iv).

By the above method the compound of interest is characterized for each of the binary binding systems used, i.e. the fluorescent-labelled fatty acid, the fatty acid binding compound and the binding and FRET-conditions used in each method. The resulting signal, i.e. the fluorescence intensity and thus the binding affinity will vary with each binding system employed and the FI and calculated binding affinity will be characteristic for the compound of interest.

Consequently, the method of the invention provides data that is characteristic for the compound of interest and which can be used to distinguish the compound of interest from other compounds that can displace the fluorescent-labelled fatty acid from the fatty acid binding compound under the conditions used.

For example, HSA (human serum albumin) and BSA (bovine serum albumin) differ in amino acid composition by 24% and both albumin types will bind fatty acids. However, due to the differences in structure, the FRET signals from the binary system used will vary with the albumin type. Similarly, the fatty acid and/or the fluorescent compound of the fluorescent-labelled fatty acid can be varied and the resulting FRET signal will be affected. The same, variation of the conditions for the binding and FRET effects of the fluorescent-labelled fatty acid with the fatty acid binding compound can lead to FRET variation.

In a particular embodiment, the method of the invention is one, wherein
(I) the fluorescent compound of the fluorescent-labelled fatty acid is selected from 4-nitrobenzo-2-oxa-1,3-diazole (NBD) and 5-(dimethylamino)naphthalene-1-sulfonyl; and/or
(II) the fatty acid component of the fluorescent-labelled fatty acid is selected from the group consisting of linear or branched, saturated or unsaturated fatty acids;
(III) the fatty acid binding compound is selected from the group consisting of proteins comprising at least one tryptophan.

In one embodiment, the fatty acid component of the fluorescent-labelled fatty acid is selected from the group consisting of linear and saturated fatty acids. In one embodiment, the linear and saturated fatty acids have 2 to 25 carbon atoms. In another embodiment, the linear and saturated fatty acids have 8 to 20 carbon atoms. In another embodiment, the linear and saturated fatty acids have 10 to 18 carbon atoms. In yet another embodiment, the linear and saturated fatty acids have 10 to 16 carbon atoms.

In one embodiment, the fatty acid binding compound is selected from the group consisting of albumin, HDL, LDL and VLDL. In one embodiment, said albumin is human or bovine serum albumin.

For example, in a particular embodiment of the method of the invention the method of the invention is performed for the compound of interest for at least the following four binary systems:
(1) human serum albumin/NBD-fatty acid,
(2) human serum albumin/dansyl-fatty acid,
(3) bovine serum albumin/NBD-fatty acid, and
(4) bovine serum albumin/dansyl-fatty acid.

Here, the fatty acid binding compound and the fluorescent compound are both varied.

In another particular embodiment of the method of the invention, the method of the invention is performed for the compound of interest for at least the following four binary systems:
(1) human serum albumin/NBD-fatty acid¹
(2) human serum albumin/NBD-fatty acid²,
(3) bovine serum albumin/NBD-fatty acid¹, and
(4) bovine serum albumin/NBD-fatty acid²,
   wherein NBD-fatty acid¹ and NBD-fatty acid² elicit different FRET signals upon binding to human serum albumin and bovine serum albumin. Here, the fatty acid binding compound and the fatty acid of the fluorescent-labelled fatty acid are both varied.

In a further particular embodiment of the method of the invention the method of the invention is performed at least eight times for the compound of interest for at least the following eight binary systems:
(1) human serum albumin/NBD-fatty acid¹
(2) human serum albumin/NBD-fatty acid²,
(3) bovine serum albumin/NBD-fatty acid¹, and
(4) bovine serum albumin/NBD-fatty acid²,
(5) human serum albumin/dansyl-fatty acid¹
(6) human serum albumin/dansyl-fatty acid²,
(7) bovine serum albumin/dansyl-fatty acid¹, and
(8) bovine serum albumin/dansyl-fatty acid²,

Here, the fatty acid binding compound, the fatty acid and the fluorescent of fluorescent-labelled fatty acid are all varied.

Variation in the binding conditions can further extend the options for binary systems suitable for characterising the compound of interest.

For identifying and/or characterizing the compound of interest, either the fluorescence signal of each binary system is used directly or it is further converted into a fluorescence-dependent parameter such as the binding affinity. The result of each binary system used in the method of the invention is characteristic for the compound of interest and can be used alone or in combination to characterize and/or identify the compound of interest relative to data obtained for known compounds that are capable of displacing the fatty acid of the fluorescent-labelled fatty acid from the fatty acid binding compound of the binary system(s) used.

In particular embodiments, the method of the present invention is used to identify and/or characterize mono-, di- and triglycerides. In particular, the method can be used to distinguish mono-, di- and triglycerides based on differences in chain length and degree as well as type of saturation. It can be used for distinguishing enantiomers. It also has utility for identifying and characterizing complex compositions, e.g. food and feed products and components thereof. A food product or feed product as used herein can be any a substance or combination of substances that can be used or prepared for use as food (such as human nutrition) or feed (such as animal nutrition), wherein, in one embodiment, the terms food or feed as used herein relate to any substance or combination of substances that can be metabolized by an animal to give energy and build tissue. In one embodiment, the animal is a human being. Particular examples comprise fats or lipids such as olive oil, sunflower oil, peanut oil, or any food or feed product that can comprise any of these oils such as fat- or oil-comprising food or feed, e.g. chocolate-comprising food products, cereals etc., beverages such as wine, e.g. red wine based on their ingredients, e.g. flavones.

In a further aspect, the present invention pertains to a kit of parts comprising at least (i) a fluorescent-labelled fatty acid and (ii) a fatty acid binding compound, wherein (a) the fatty acid component of the fluorescent-labelled fatty acid binds the fatty acid binding compound and (b) the fluorescent component of the fluorescent-labelled fatty acid and the fatty acid binding compound interact to elicit FRET (Förster resonance energy transfer) effects.

In the context of the present invention, a kit of parts (in short: kit) is understood to be any combination of the components identified in this application, which are combined, coexisting spatially, to a functional unit, and which can contain further components.

Optionally, the kit of parts further comprises one or more of the following:
a) a data carrier (such as an instruction manual, leaflet, label, tag, chip or bar code) comprising e.g. handling information, storage information (e.g. storage conditions such as temperature), safety-information, instructions for carrying out one or more of the methods of present invention, batch or lot number, expiry date of the kit or one or more of its constituents
b) one or more containers or packages or packaging material
c) one or more solutions, buffers and/or other compounds and compositions useful for performing a method of the present invention.

In one embodiment, in the kit
(i) the fluorescent component is selected from the group consisting of 4-nitrobenzo-2-oxa-1,3-diazole (NBD) and 5-(dimethylamino)naphthalene-1-sulfonyl (dansyl) and/or
(ii) the fatty acid component is selected from the group consisting of linear or branched, saturated or unsaturated fatty acids, and/or
(iii) the fatty acid binding compound is selected from the group consisting of proteins comprising at least one tryptophan.

In one embodiment, the fatty acid component of the fluorescent-labelled fatty acid is selected from the group consisting of linear and saturated fatty acids. In one embodiment, the linear and saturated fatty acids have 2 to 25 carbon atoms. In another embodiment, the linear and saturated fatty acids have 8 to 20 carbon atoms. In another embodiment, the linear and saturated fatty acids have 10 to 18 carbon atoms. In yet another embodiment, the linear and saturated fatty acids have 10 to 16 carbon atoms.

In one embodiment, the fatty acid binding compound is selected from the group consisting of albumin, HDL, LDL and VLDL. In one embodiment, said albumin is human or bovine serum albumin.

In a particular embodiment, the kit of the invention is one, wherein
(i) the fluorescent component is 4-nitrobenzo-2-oxa-1,3-diazole (NBD),
(ii) the fatty acid component is selected from the group consisting of linear and saturated fatty acids having 8 to 25 carbon atoms
(iii) the fatty acid binding compound is albumin.

In one embodiment, the linear and saturated fatty acids have 8 to 20 carbon atoms. In another embodiment, the linear and saturated fatty acids have 10 to 18 carbon atoms. In yet another embodiment, the linear and saturated fatty acids have 10 to 16 carbon atoms.

In one embodiment, the fatty acid binding compound is human serum albumin.

In one embodiment, the kit of the invention comprises instructions for performing at least one of steps (i) to (vi). In a particular embodiment, the kit of the invention comprises instructions for performing at least steps (iii) and/or (iv) of the method of the invention.

An additional aspect of the present invention relates to the use of a composition of the invention and/or a kit of parts of the invention for performing a method of the invention. Furthermore, the invention is useful for identifying and/or characterizing mono, di- and triglycerides as well as for identifying and/or characterizing food and feed products.

In a further aspect, present invention relates to a method of manufacturing a composition of present invention comprising admixing a fluorescent-labelled fatty acid and a fatty acid binding compound under conditions allowing for a binding of the fatty acid component of the fluorescent-labelled fatty acid and the fatty acid binding compound. The labelling of the fatty acid and exemplary conditions suitable for the binding of the fatty acid binding compound and the fluorescent-labelled fatty acid are described in the example section and can be performed by the skilled artisan on basis of said description and the general knowledge without undue burden.

In a sixth aspect, present invention relates to a method of manufacturing a kit of present invention comprising assembling the different components of the kit to form a spatial and/or functional unit. In one embodiment, the method further comprises packaging the different components into one or more containers. The packaging and assembly can be performed according to standard procedures, e.g. allowing for safe and long-term storage of the individual components.

The different aspects of present invention and their embodiments can be combined with each other. In addition, any of the aspects and their embodiments described above can be combined with any of the particular embodiments as listed herein below.

Some particular embodiments that further serve to illustrate the present invention are given in the following:

### DESCRIPTION OF EMBODIMENTS

1. Composition comprising
   (i) a fluorescent-labelled fatty acid and
   (ii) a fatty acid binding compound,

wherein
(a) the fatty acid component of the fluorescent-labelled fatty acid binds the fatty acid binding compound and
(b) the fluorescent component of the fluorescent-labelled fatty acid and the fatty acid binding compound interact to elicit FRET (Förster resonance energy transfer) effects.
2. Composition according to aspect 1, wherein the fluorescent component of the fluorescent-labelled fatty acid is selected from the group consisting of 4-nitrobenzo-2-oxa-1,3-diazole (NBD) and 5-(dimethylamino)naphthalene-1-sulfonyl (dansyl).
3. Composition according to aspects 1 or 2, wherein the fatty acid component of the fluorescent-labelled fatty acid is selected from the group consisting of linear or branched, saturated or unsaturated fatty acids.
4. Composition according to aspect 3, wherein the fatty acid component of the fluorescent-labelled fatty acid is selected from the group consisting of linear and saturated fatty acids having 2 to 25 carbon atoms.
5. Composition according to any of aspects 1 to 4, wherein the fatty acid binding compound is selected from the group consisting of proteins comprising at least HDL, LDL and VLDL.
6. Composition according to any of aspects 1 to 5, wherein
   (i) the fluorescent component is 4-nitrobenzo-2-oxa-1,3-dizole (NBD),
   (ii) the fatty acid component is selected from the group consisting of linear and saturated fatty acids having 8 to 25 carbon atoms, and
   (iii) the fatty acid binding compound is albumin.
7. Composition according to aspect 6, wherein the concentration of the fluorescent-labelled fatty acid is 2 μM to 50 μM.
8. Method for identifying and/or characterizing a compound of interest, comprising the steps of:
   (i) providing a fluorescent-labelled fatty acid,
   (ii) providing a fatty acid binding compound that binds to the fatty acid and interacts with the fatty acid to elicit FRET (Förster resonance energy transfer) effects,
   (iii) contacting the fluorescent-labelled fatty acid with the fatty acid binding compound under conditions that allow for (a) the binding of the fatty acid to the fatty acid binding compound and for (b) FRET (Förster resonance energy transfer) effects,
   (iv) contacting the fluorescent-labelled fatty acid bound to the fatty acid binding compound with a compound of interest under conditions that allow for (a) the binding of the fatty acid to the fatty acid binding compound and for (b) FRET (Förster resonance energy transfer) effects,
   (v) determining the change in fluorescence, and
   (vi) optionally calculating the binding affinity of the compound of interest to the fatty acid binding compound.
9. Method according to aspect 8, wherein the fluorescent component of the fluorescent-labelled fatty acid is selected from the group consisting of 4-nitrobenzo-2-oxa-1,3-diazole (NBD) and 5-(dimethylamino)naphthalene-1-sulfonyl (dansyl).
10. Method according to aspects 8 or 9, wherein the fatty acid component of the fluorescent-labelled fatty acid is selected from the group consisting of linear or branched, saturated or unsaturated fatty acids.
11. Method according to aspect 10, wherein the fatty acid component of the fluorescent-labelled fatty acid is selected from the group consisting of linear and saturated fatty acids having 2 to 25 carbon atoms.
12. Method according to any of aspects 8 to 11, wherein the fatty acid binding compound is selected from the group consisting of proteins comprising at least one tryptophan.
13. Method according to any of aspects 8 to 12, wherein
   (i) the fluorescent component is 4-nitrobenzo-2-oxa-1,3-dizole (NBD),
   (ii) the fatty acid component is selected from the group consisting of linear and saturated fatty acids having 8 to 25 carbon atoms,
   (iii) the fatty acid binding compound is albumin.
14. Method according to aspect 13, wherein the concentration of the fluorescent-labelled fatty acid is 2 to 50.
17. Method according to aspect 15 or 16, wherein the method is performed for the compound of interest for at least the following four binary systems:
   (1) human serum albumin/NBD-fatty acid,
   (2) human serum albumin/dansyl-fatty acid,
   (3) bovine serum albumin/NBD-fatty acid, and
   (4) bovine serum albumin/dansyl-fatty acid.
18. Method according to any one of aspects 15 to 17, wherein the method is performed for the compound of interest for at least the following four binary systems:
   (1) human serum albumin/NBD-fatty acid¹
   (2) human serum albumin/NBD-fatty acid²,
   (3) bovine serum albumin/NBD-fatty acid¹, and
   (4) bovine serum albumin/NBD-fatty acid²,
   wherein NBD-fatty acid¹ and NBD-fatty acid² elicit different FRET signals upon binding to human serum albumin and bovine serum albumin.
19. Method according to any one of aspects 15 to 18, wherein the method is performed for the compound of interest for at least the following eight binary systems:
   (1) human serum albumin/NBD-fatty acid¹
   (2) human serum albumin/NBD-fatty acid²,
   (3) bovine serum albumin/NBD-fatty acid¹, and
   (4) bovine serum albumin/NBD-fatty acid²,
   (5) human serum albumin/dansyl-fatty acid¹
   (6) human serum albumin/dansyl-fatty acid²,
   (7) bovine serum albumin/dansyl-fatty acid¹, and
   (8) bovine serum albumin/dansyl-fatty acid²,
   wherein NBD-fatty acid¹ and NBD-fatty acid² elicit different FRET signals upon binding to human serum albumin and bovine serum albumin.
20. Kit of parts comprising at least
   (i) a fluorescent-labelled fatty acid and
   (ii) a fatty acid binding compound,
   wherein
   (a) the fatty acid component of the fluorescent-labelled fatty acid binds the fatty acid binding compound and
   (b) the fluorescent component of the fluorescent-labelled fatty acid and the fatty acid binding compound interact to elicit FRET (Förster resonance energy transfer) effects.
21. Kit according to aspect 20, wherein
   (i) the fluorescent component is selected from the group consisting of 4-nitrobenzo-2-oxa-1,3-diazole (NBD) and 5-(dimethylamino)naphthalene-1-sulfonyl (dansyl) and/or
   (ii) the fatty acid component is selected from the group consisting of linear or branched, saturated or unsaturated fatty acids, and/or
   (iii) the fatty acid binding compound is selected from the group consisting of proteins comprising at least one tryptophan.
22. Kit according to any of aspects 20 and 21, wherein
   (i) the fluorescent component is 4-nitrobenzo-2-oxa-1,3-diazole (NBD),
   (ii) the fatty acid component is selected from the group consisting of linear and saturated fatty acids having 2 to 25 carbon atoms,
   (iii) the fatty acid binding compound is albumin.
23. Kit according to aspect 22, comprising instructions for performing at least one of steps (i) to (vi) of the method of aspect 8.
24. Use of a composition according to any one of aspects 1 to 7 and/or a kit of parts according to any of aspects 20 to 23 for performing a method according to any of aspects 8 to 19.
25. Use according to aspect 24 for identifying and/or characterizing mono-, di- and triglycerides.
26. Use according to aspect 24 for identifying and/or characterizing food and feed products.
27. Method of manufacturing a composition according to one of the aspects 1 to 7 comprising admixing the fluorescent-labelled fatty acid and the fatty acid binding compound under conditions allowing for a binding of the fatty acid component of the fluorescent-labelled fatty acid and the fatty acid binding compound.
28. Method of manufacturing a kit according to one of the aspects 20 to 23 comprising assembling the different components of the kit to form a spatial and/or functional unit.

The following examples represent embodiments presented for the sole purpose of illustrating the present invention and are by no means to be interpreted as limiting the scope of the appended claims.

### EXAMPLES

### General Methods

### Chromatographic Methods

Glass plates coated with silica gel 60 F₂₅₄ (Merck) were used for thin-layer chromatography (TLC).

Preparative reversed phase (RP-)HPLC was carried out using an acetonitrile/water eluent. Separations without trifluoroacetic acid (TFA) were carried out on a Waters Pump 2525 HPLC with a column from Waters of the SunFire™ Prep C₁₈ type (10 μm, 50×250 mm). Separations with TFA were carried out on an Agilent 1100 Series HPLC with an Agilent Prep. C₁₈ column (10 μm, 30×250 mm). The gradients used are shown below as Method A and Method B. Normal-phase chromatography on silica gel was carried out using a preparative automated chromatograph, the Isolera One from Biotage. Prepacked columns of the type Biotage SNAP Cartridge KP-Sil (10 g) were used. Two different eluent gradients were utilized (Method C and Method D).

### Method A:

| | Time | | |
|---|---|---|---|
| (RP-HPLC without TFA) | [min] | Acetonitrile [%] | Water [%] |
| | 0 | 10 | 90 |
| | 4 | 10 | 90 |
| | 24 | 80 | 20 |
| | 28 | 10 | 90 |
| | 35 | 10 | 90 |

| | |
|---|---|
| Flow rate | 150 ml/min |
| Injection volume | 2500-5000 μl |

### Method B:

| | Time | | Water [%] |
|---|---|---|---|
| (RP-HPLC with TFA) | [min] | Acetonitrile [%] | (0.1 % TFA) |
| | 0 | 10 | 90 |
| | 12.5 | 90 | 10 |
| | 14 | 90 | 10 |
| | 14.5 | 10 | 90 |
| | 16 | 10 | 90 |

| | |
|---|---|
| Flow rate | 80 ml/min |
| Injection volume | 5000 μl |

### Method C:

| | Time | | |
|---|---|---|---|
| (NP-HPLC) | [min] | DCM [%] | MeOH [%] |
| | 0 | 95 | 5 |
| | 2.4 | 95 | 5 |
| | 12.5 | 68 | 32 |
| | 16.1 | 60 | 40 |
| | 21.4 | 60 | 40 |

| | |
|---|---|
| Wavelength | 254 nm, 366 nm |
| Flow rate | 12 ml/min |

### Method D:

| | Time | | |
|---|---|---|---|
| (NP-HPLC) | [min] | Hep [%] | EA [%] |
| | 0 | 95 | 5 |
| | 2.3 | 95 | 5 |
| | 12.3 | 50 | 50 |
| | 17.2 | 50 | 50 |

| | |
|---|---|
| Wavelength | 254 nm, 366 nm |
| Flow rate | 12 ml/min |

### Spectroscopic Methods

Absorption and fluorescence of fluorescence-labelled derivatives and HSA were carried out in a Varioskan™ microplate reader (Thermo Electron Corporation). 96 well (Costar, half area, flat bottom) microplates were used for absorption measurements and 384 well (Greiner Bio One, small volume, black) microplates from Greiner Bio One were used for fluorescence measurements. All measurements were carried out at pH 7.4 and room temperature.

### Mass Spectrometric Methods

A 1200 Series LCMS system from Agilent Technologies with a Phenomenex Luna C₁₈(2): column (3 μm, 10×2 mm) was used for retention time and mass determination. The following method, which detects in a mass range from 110-1000 mass units, was used.

### LCMS FRA Method:

| FRA | Time | | Water [%] |
|---|---|---|---|
| method | [min] | Acetonitrile [%] | (0.05% TFA) |
| | 0 | 5 | 95 |
| | 1.2 | 95 | 5 |
| | 1.4 | 95 | 5 |
| | 1.5 | 3 | 97 |

| | |
|---|---|
| Flow rate | 1.1 ml/min |
| Injection volume | 0.2 μl |

The molar weights are indicated in [g/mol], the detected masses in mass per charge [m/e].

### Other Methods

log D_{7.4} values were determined by a method similar to the partition coefficient method in a water/octanol mixture and were carried out on an RP-HPLC from Waters Alliance (2795) having a C₁₈ column (2×20 mm) with a gradient of morpholinesulfonic acid buffer (pH 7.4) and acetonitrile. The range was between −1 (hydrophobic) to +6 (lipophilic).

### Example 1

### Synthesis of Fluorescence-Labelled Fatty Acids

For binding experiments with fluorescence-labelled fatty acids two different dyes, NBD and dansyl were selected. Aliphatic unbranched fatty acids with a chain length of C₂ to C₁₂ were coupled to the two dye labels by nucleophilic substitution. Dansyl was additionally coupled to methylamine hydrochloride.

### 1.1 Preparation of NBD-Labelled Derivatives

71 mg (0.36 mmol) 4-chloro-7-nitrobenzo-2-oxa-1,3-diazole (1) and 90 mg (1.07 mmol) NaHCO₃ were added to a solution of 0.36 mmol of the appropriate amino acid (6-11) in 15 ml MeOH. The reaction mixture was then heated to reflux at 80° C. with the exclusion of light for 3.5 hours. The solution was cooled to room temperature and the solvent was removed under reduced pressure. The residue was dissolved in 20 ml water and adjusted to pH 3 with 0.5 M HCl. The aqueous solution was extracted 3× with 20 ml ethyl acetate. The combined organic phases were dried with MgSO₄, filtered and the solvent was distilled under reduced pressure. The residue was purified using the method indicated. Reaction monitoring was by HPLC/MS (FRA method). The following compounds were prepared according to this general experimental procedure.

TABLE 1
NBD fluorescence-labelled compounds

| | | | Yield |
|---|---|---|---|
| Ref. no. | Starting material | Product | [%] |
| 1 | Aminoethanoic acid (6) | 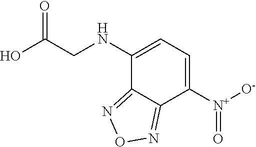 [SMILES: O=C(O)CNc1ccc([N+](=O)[O-])c2nonc12] | 24 |
| | | (12) | |
| 2 | 4-Aminobutanoic acid (7) | 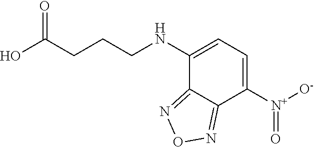 [SMILES: O=C(O)CCCNc1ccc([N+](=O)[O-])c2nonc12] | 43 |
| | | (13) | |
| 3 | 6-Aminohexanoic acid (8) | 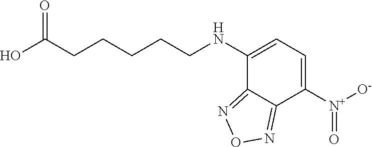 [SMILES: O=C(O)CCCCCNc1ccc([N+](=O)[O-])c2nonc12] | 47 |
| | | (14) | |
| 4 | 8-Aminooctanoic acid (9) | 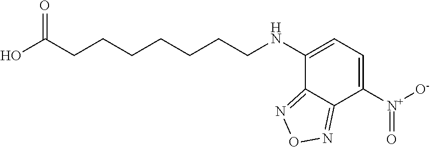 [SMILES: O=C(O)CCCCCCCNc1ccc([N+](=O)[O-])c2nonc12] | 37 |
| | | (15) | |
| 5 | 10-Amindecanoic acid (10) | 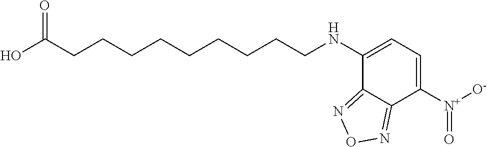 [SMILES: O=C(O)CCCCCCCCCNc1ccc([N+](=O)[O-])c2nonc12] | 40 |
| | | (16) | |
| 6 | 12-Aminododecanoic acid (11) | 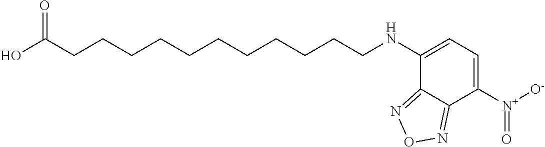 [SMILES: O=C(O)CCCCCCCCCCCNc1ccc([N+](=O)[O-])c2nonc12] | 36 |
| | | (17) | |

Specifically, NBD fluorescence-labelled compounds were prepared as follows:

### 2-(7-nitrobenzo[1,2,5]oxadiazol-4-ylamino)ethanoic acid (12)

399 mg (1.99 mmol) NBD-Cl (1) and 504 mg (5.99 mmol) NaHCO₃ were added to a solution of 150 mg (1.99 mmol) of aminoethanoic acid (6) in 40 ml MeOH. The reaction time was 3 h at 80° C. with exclusion of light. The residue was purified using method B (RP-HPLC with TFA). Ion chromatography showed that no TFA from the HPLC separation and also no other cations or anions were contained in the substance. Yield: 115 mg (0.48 mmol, 24%), Rₜ 0.93 min, orange-colored solid, M.p. 170-175° C. (dec.); log D_{7.4}<−1, MS (ES+) 239.04 [M+H], calculated for C₈H₆N₄O₅ 238.16.

### 4-(7-Nitrobenzo[1,2,5]oxadiazol-4-ylamino)butanoic acid (13)

193.6 mg (0.97 mmol) NBD-Cl (1) and 244.5 mg (2.91 mmol) NaHCO₃ were added to a solution of 100 mg (0.97 mmol) of 4-amino-butanoic acid (7) in 35 ml of MeOH. The reaction time was 3 h at 80° C. with exclusion of light. The residue was purified using method A (RP-HPLC). Yield: 110 mg (0.41 mmol, 43%), Rₜ 1.36 min, orange-colored solid, M.p. 196.5° C.; log D_{7.4}=−0.46, MS (ES+) 267.07 [M+H], calculated for C₁₀H₁₀N₄O₅ 266.22.

### 6-(7-Nitrobenzo[1,2,5]oxadiazol-4-ylamino)hexanoic acid (14)

152 mg (0.76 mmol) NBD-Cl (1) and 192 mg (2.28 mmol) NaHCO₃ were added to a solution of 100 mg (0.76 mmol) of 6-aminohexanoic acid (8) in 30 ml of MeOH. The reaction time was 3 h at 80° C. with exclusion of light. The residue was purified using method B (RP-HPLC with TFA). Ion chromatography showed that about 1 eq. of TFA from the HPLC separation was contained in the substance. Yield: 62 mg (0.21 mmol, 47%), Rₜ=1.59 min, orange-brown solid, M.p. 245-250° C. (dec.); log D_{7.4} 0.53, MS (ES+) 295.1 [M+H], calculated for C₁₂H₁₄N₄O₅ 294.27.

### 8-(7-Nitrobenzo[1,2,5]oxadiazol-4-ylamino)octanoic acid (15)

125.3 mg (0.63 mmol) NBD-Cl (1) and 158.3 mg (1.88 mmol) NaHCO₃ were added to a solution of 100 mg (0.63 mmol) of 8-amino-octanoic acid (9) in 27 ml of methanol. The reaction time was 3 h at 80° C. with exclusion of light. The residue was purified using method A (RP-HPLC). Yield: 54 mg (0.17 mmol, 27%), Rₜ=1.75 min, orange solid, M.p. 155.8° C.; log D_{7.4} 1.29, MS (ES+) 323.13 [M+H], calculated for C₁₄H₁₈N₄O₅=322.32.

### 10-(7-Nitrobenzo[1,2,5]oxadiazol-4-ylamino)decanoic acid (16)

106.6 mg (0.53 mmol) of NBD-CI (1) and 134.6 mg (1.6 mmol) NaHCO₃ were added to a solution of 100 mg (0.53 mmol) of 10-aminodecanoic acid (10) in 22 ml MeOH. The reaction time was 3 h at 80° C. with exclusion of light. The residue was purified by method A (RP-HPLC). Yield: 74 mg (0.21 mmol, 40%), Rₜ=1.89 min, orange-colored solid, M.p. 136.4° C.; log D_{7.4}=2.03, MS (ES+) 351.16 [M+H], calculated for C₁₆H₂₂N₄O₅ 350.38.

### 12-(7-Nitrobenzo[1,2,5]oxadiazol-4-ylamino)dodecanoic acid (17)

92.6 mg (0.46 mmol) NBD-CI (1) and 117 mg (1.39 mmol) NaHCO₃ were added to a solution of 100 mg (0.46 mmol) 12-aminododecanoic acid (11) in 20 ml MeOH. Reaction time was 3 h at 80° C. with exclusion of light. The residue was purified using method A (RP-HPLC). Yield: 60 mg (0.16 mmol, 36%), Rₜ=2.01 min, orange-colored solid, M.p. 113.4° C.; log D_{7.4}=2.84, MS (ES+) 379.19 [M+H], calculated for C₁₈H₂₆N₄O₅=378.43.

### 1.2 Preparation of Dansyl-Labelled Derivatives

3.78 g (45 mmol) of NaHCO₃ were added to a solution of 11.4 mmol of the amino acid/amine (6-11/18) in 45 ml water. A solution of 0.63 g (2.33 mmol) of 5-(dimethylamino)naphthalene-1-sulfonyl chloride (4) in 10 ml acetone and 2.00 ml (14.43 mmol) triethylamine was slowly added dropwise to the mixture. The reaction was stirred for 2 hours at room temperature with exclusion of light. The solution was acidified with 0.5 M HCl to pH 3 and extracted 3× with 30 ml ethyl acetate. The combined organic phases were dried using MgSO₄, filtered and the solvent was removed under reduced pressure. The residue was purified using the method indicated in each case. Reaction control was by HPLC/MS (FRA method). The following compounds were prepared by this procedure.

TABLE 2
NBD fluorescence-labelled compounds

| | | | Yield |
|---|---|---|---|
| Rf. No. | Starting material | Product | [%] |
| 1 | Aminoethanoic acid (6) | 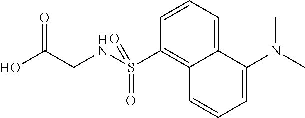 [SMILES: CN(C)c1cccc2c(S(=O)(=O)NCC(=O)O)cccc12] | 61 |
| | | (19) | |
| 2 | 4-Aminobutanoic acid (7) | 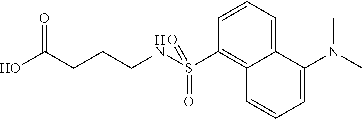 [SMILES: CN(C)c1cccc2c(S(=O)(=O)NCCCC(=O)O)cccc12] | 50 |
| | | (20) | |
| 3 | 6-Aminohexanoic acid (8) | 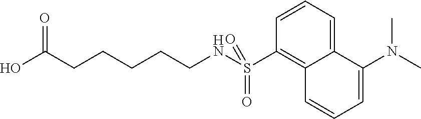 [SMILES: CN(C)c1cccc2c(S(=O)(=O)NCCCCCC(=O)O)cccc12] | 49 |
| | | (21) | |
| 4 | 8-Aminooctanoic acid (9) | 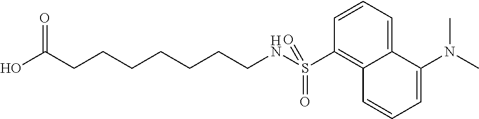 [SMILES: CN(C)c1cccc2c(S(=O)(=O)NCCCCCCCC(=O)O)cccc12] | 91 |
| | | (22) | |
| 5 | 10-Aminodecanoic acid (10) | 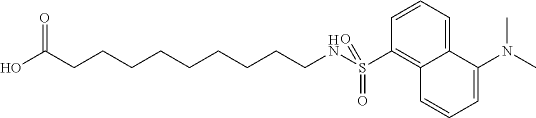 [SMILES: CN(C)c1cccc2c(S(=O)(=O)NCCCCCCCCCC(=O)O)cccc12] | 81 |
| | | (23) | |
| 6 | 12- Aminododecanoic acid (11) | 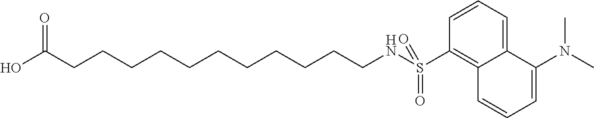 [SMILES: CN(C)c1cccc2c(S(=O)(=O)NCCCCCCCCCCCC(=O)O)cccc12] | 99 |
| | | (24) | |
| 7 | Methylamine hydrochloride (18) | 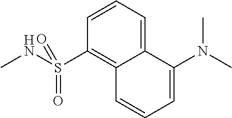 | 92 |
| | | (25) | |

Specifically, NBD fluorescence-labelled compounds were prepared as follows:

### (5-Dimethylaminonaphthalene-1-sulfonylamino)ethanoic acid (19)

1.185 g (14.11 mmol) NaHCO₃ and 200 mg (0.74 mmol) dansyl Cl (4) in 4 ml acetone were added to a solution of 265 mg (3.53 mmol) aminoethanoic acid (6) in 14 ml water. 638 μl (4.59 mmol) triethylamine were finally added dropwise. The reaction time was 2 h at RT with exclusion of light. The residue was purified using method C (NP-HPLC), yield: 140 mg (0.45 mmol, 61%), Rₜ 1.39 min, yellow oil, log D_{7.4} 1.02, MS (ES+) 309.08 [M+H], calculated for C₁₄H₁₆N₂O₄S 308.36.

### 4-(5-Dimethylaminonaphthalene-1-sulfonylamino)butanoic acid (20)

594 mg (7.07 mmol) NaHCO₃ and 100 mg (0.37 mmol) of dansyl Cl (4) in 1.8 ml of acetone were added to a solution of 182.2 mg (1.77 mmol) of 4-aminobutanoic acid (7) in 7 ml water. 319 μl (2.3 mmol) triethylamine were finally added dropwise. The reaction time was 2 h at RT with exclusion of light. The residue was purified using method A (RP-HPLC), yield: 62 mg (0.18 mmol, 50%), Rₜ 1.64 min, yellow-brownish oil, log D_{7.4} 1.21, MS (ES+) 337.11 [M+H], calculated for C₁₆H₂₀N₂O₄S 336.41.

### 6-(5-Dimethylaminonaphthalene-1-sulfonylamino)hexanoic acid (21)

2.56 g (30.5 mmol) NaHCO₃ and 432 mg (1.6 mmol) dansyl Cl (4) in 7 ml acetone were added to a solution of 1 g (7.62 mmol) 6-amino-hexanoic acid (8) in 30 ml water. 1378 μl (9.91 mmol) of triethylamine were finally added dropwise. The reaction time was 2 h at RT with exclusion of light. The residue was purified using method A (RP-HPLC), yield: 242 mg (0.66 mmol, 49%), R₁ 1.77 min, green-yellow oil, log D_{7.4} 1.56, MS (ES+) 365.15 [M+H], calculated for C₁₈H₂₄N₂O₄S 364.47.

### 8-(5-Dimethylaminonaphthalene-1-sulfonylamino)octanoic acid (22)

594 mg (7.07 mmol) NaHCO₃ and 100 mg (0.37 mmol) dansyl Cl (4) in 1.8 ml acetone were added to a solution of 281.4 mg (1.77 mmol) 8-aminooctanoic acid (9) in 7 ml water. 319 μl (2.3 mmol) triethylamine were finally added dropwise. The reaction time was 2 h at RT with exclusion of light. The residue was purified using method A (RP-HPLC), yield: 133 mg (0.34 mmol, 91%), Rₜ 1.89 min, brown oil, log D_{7.4} 2.1, MS (ES+) 393.18 [M+H], calculated for C₂₀H₂₈N₂O₄S 392.52.

### 10-(5-Dimethylaminonaphthalene-1-sulfonylamino)decanoic acid (23)

594 mg (7.07 mmol) NaHCO₃ and 100 mg (0.37 mmol) dansyl Cl (4) in 1.8 ml acetone were added to a solution of 331 mg (1.77 mmol) 10-aminodecanoic acid (10) in 7 ml water. 319 μl (2.3 mmol) triethylamine were finally added dropwise. The reaction time was 2 h at RT with exclusion of light. The residue was purified using method A (RP-HPLC), yield: 127 mg (0.3 mmol, 81%), Rₜ 2.01 min, brown oil, log D_{7.4}=2.74, MS (ES+) 421.21 [M+H], calculated for C₂₂H₃₂N₂O₄S=420.58.

### 12-(5-Dimethylaminonaphthalene-1-sulfonylamino)dodecanoic acid (24)

594 mg (7.07 mmol) NaHCO₃ and 100 mg (0.37 mmol) of dansyl Cl (4) in 1.8 ml of acetone were added to a solution of 380.5 mg (1.77 mmol) of 12-aminododecanoic acid (11) in 7 ml of water. 319 μl (2.3 mmol) of triethylamine are finally added dropwise. The reaction time was 2 h at RT with exclusion of light. The residue was purified using method A (RP-HPLC), yield: 164 mg (0.37 mmol, 99%), Rₜ 2.11 min, light brown oil, log D_{7.4} 3.49, MS (ES+) 449.24 [M+H], calculated for C₂₄H₃₆N₂O₄S 448.63.

### 5-Dimethylaminonaphthalene-1-sulfonic acid methylamide (25)

1.185 g (14.11 mmol) NaHCO₃ and 200 mg (0.74 mmol) dansyl Cl (4) in 4 ml of acetone were added to a solution of 238.3 mg (3.53 mmol) of methylamine hydrochloride (18) in 14 ml of water. 638 μl (4.59 mmol) of triethylamine ware finally added dropwise. The reaction time was 2 h at RT with exclusion of light. The residue was purified using method D (NP-HPLC), yield: 180 mg (0.68 mmol, 92%), Rₜ 1.58 min, yellow oil, log D_{7.4} 2.76, MS (ES+) 265.09 [M+H], calculated for C₁₃H₁₆N₂O₄S 264.35.

### Example 2

### Stock Solutions

For investigating the binding of the dyes synthesized in Example 1 to HSA, absorption and fluorescence spectroscopy methods were employed. The individual dyes were measured at different concentrations to establish their spectroscopic characteristics. For both absorption and fluorescence measurement the NBD compounds showed a greater spectral absorption compared to the corresponding dansyl dyes. For both dye classes concentrations within the linear measuring range were selected, i.e a concentration of 25 μM for NBD derivatives and 100 μM for dansyl derivatives.

A 10 μM stock solution for all synthesized fluorescence-labelled compounds in DMSO was prepared. The following tables show the respective initial weights of the substances and the volumes of DMSO used.

### Structure of the Dye Label NBD:

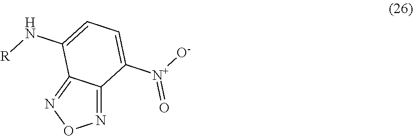

TABLE 3
NBD stock solutions

| | Molar mass | | 10 mM stock |
|---|---|---|---|
| —R | [g/mol] | abbreviation | solution |
| —CH₂COOH (12) | 238.16 | NBD C₂ | 4 mg + 1680 μl DMSO |
| —C₃H₆COOH (13) | 266.22 | NBD C₄ | 5 mg + 1878 μl DMSO |
| —C₅H₁₀COOH (14) | 294.27 | NBD C₆ | 5 mg + 1699 μl DMSO |
| —C₇H₁₄COOH (15) | 322.32 | NBD C₈ | 6 mg + 1862 μl DMSO |
| —C₉H₁₈COOH (16) | 350.38 | NBD C₁₀ | 6 mg + 1712 μl DMSO |
| —C₁₁H₂₂COOH (17) | 378.43 | NBD C₁₂ | 7 mg + 1850 μl DMSO |

### Structure of the Dye Label Dansyl:

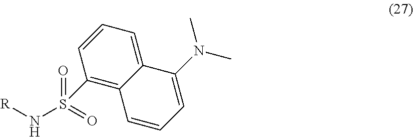

TABLE 4
Dansyl stock solutions

| | Molar mass | | |
|---|---|---|---|
| —R | [g/mol] | abbreviation | 10 mM stock solution |
| —CH₂COOH (19) | 308.36 | dansyl C₂ | 6 mg + 1946 μl DMSO |
| —C₃H₆COOH (20) | 336.41 | dansyl C₄ | 6 mg + 1784 μl DMSO |
| —C₅H₁₀COOH (21) | 364.47 | dansyl C₆ | 7 mg + 1921 μl DMSO |
| —C₇H₁₄COOH (22) | 392.52 | dansyl C₈ | 7 mg + 1783 μl DMSO |
| —C₉H₁₈COOH (23) | 420.58 | dansyl C₁₀ | 8 mg + 1902 μl DMSO |
| —C₁₁H₂₂COOH | 448.63 | dansyl C₁₂ | 8 mg + 1783 μl DMSO |
| (24) | | | |

The HSA (Sigma Aldrich, A1887) had ≦0.007% fatty acids. For preparing a 1515 μM HSA stock solution, 1 g HSA was combined with 9929 μl ultrapure water. The solution was aliquoted at 500 μl into Eppendorf vessels and stored at −25° C. until use.

Dilution series for competition experiments with the fatty acids C₆ to C₁₆ were diluted with phosphate-buffered saline solution (DPBS buffer). Geometric dilution series of each fatty acid were prepared in 384 well microplates with a starting concentration of 2500 μM and a dilution factor of 0.33 and also 9 dilution steps. The final volume of each concentration was 80 μl. The plated-out fatty acids were stored in the deep freeze (−25° C.) until use.

In each case, a 30 μM stock solution in DMSO was prepared for the competition experiments with ibuprofen and warfarin. The ibuprofen solution was prepared from 6 mg of ibuprofen in 970 μl of DMSO. For the warfarin solution, 10 mg were dissolved in 1081 μl of DMSO. All subsequent dilution steps of the stock solutions for the spectroscopic measurements were carried out with DPBS buffer (pH 7.4) (Invitrogen).

### Example 3

### Absorption/Excitation Wavelength and the Emission Maxima of Fluorescence-Labelled Fatty Acids

The absorption or excitation wavelength and the emission maxima of the individual compounds were determined and compared to values established in preliminary experiments. NBD derivatives feature an excitation wavelength of 480 nm and an emission maximum at 550 nm. Dansyl dyes feature an excitation wavelength of 330 nm and an emission maximum at 560 nm.

3.1 Solutions and Conditions for Absorption and Fluorescence of the Fluorescence-Labelled Derivatives without HSA

1.00 ml of a 200 μM solution was prepared from the dye stock solutions listed in Tables 3 and 4. For each dye derivative a geometric dilution series with 7 dilution steps and a dilution factor of 0.5 was prepared such that a final volume of each concentration of 500 μl was provided. From each dilution series 100 μl were pipetted into a 96 well microplate.

The conditions for the absorption measurements were:
wavelength 480 nm for NBD derivatives (12-17),
wavelength 330 nm for dansyl derivatives (19-25),
wavelength scan from 250 nm to 700 nm.

For fluorescence measurements the solutions from the 96 well microplate were used. For each dilution series 15 μl were pipetted into a 384 well microplate 4 times (fourfold determination).

The conditions for the emission measurement were:
wavelengths: 480-550 nm for NBD derivatives (12-17),
330-560 for the dansyl derivatives (19-25),
wavelength scan from 270 nm to 700 nm.
   3.2 Absorption Maxima of NBD and Dansyl Dyes without HSA

The individual absorption maxima of the concentrations chosen beforehand of 25 μM for NBD derivatives and 100 μM for dansyl derivatives were measured and compared with one another. In each case a higher concentration of both dye derivatives was measured, because the absorption maxima could be determined more accurately at higher concentrations. The higher concentrations were 50 μM for NBD derivatives and 200 μM for dansyl derivatives.

TABLE 5
Absorption maxima of the dye derivatives

| | Absorption | | Absorption |
|---|---|---|---|
| Compound | maxima [nm] | Compound | maxima [nm] |
| NBD C₂ (12) | 476 | dansyl C₂ (19) | 326 |
| NBD C₄ (13) | 482 | dansyl C₄ (20) | 326 |
| NBD C₆ (14) | 486 | dansyl C₆ (21) | 326 |
| NBD C₈ (15) | 486 | dansyl C₈ (22) | 326 |
| NBD C₁₀ (16) | 486 | dansyl C₁₀ (23) | 326 |
| NBD C₁₂ (17) | 486 | dansyl C₁₂ (24) | 322 |
| | | DMA (25) | 328 |
| Mean value | 484 | Mean value | 326 |

All absorption maxima of the individual NBD and dansyl derivatives vary little. The increasing fatty acid chain length exerts only a slight influence on the absorption maxima.

A doubling of the concentration of NBD C₁₂ (17) containing 25 μM to NBD having a concentration of 50 μM led to an approximate doubling of the absorption strength from 0.4 to 0.8. With dansyl C₁₂ (24) this concentration effect was not determinable so precisely on account of the position of the absorption maximum at shorter wavelengths. Moreover, the measuring solution of the dansyl derivative had only a weakly pronounced maximum at 100 μM.

3.3 Fluorescence of NBD and Dansyl Dyes without HSA

For the measurement of the concentration dependencies of the dyes (12-17; 19-25) the same concentrations were used as in the absorption measurement, since suitable concentrations with adequate fluorescence intensities for further experiments were also selected here. The measurement of the fluorescence using the dimensionless unit FI (fluorescence intensity) was carried out with the parameters of 480-550 nm (NBD) and 330-560 nm (dansyl) known from preliminary experiments and literature. A fourfold determination of each measuring solution was carried out and a mean value was formed.

### 3.3.1 NBD Dyes

The NBD dyes differed clearly from the dansyl dyes. The course of the curve of the NBD compounds had a hyperbolic course shape, while the dansyl compounds more nearly showed a linear to slightly hyperbolic straight course.

The fluorescence of the NBD dyes changed from a concentration of approximately 50 μM to a saturation curve, while the curves ran linearly up to a concentration of 25 μM. Between the concentrations 100 μM and 200 μM the fluorescence intensities hardly differed from one another. This saturation effect was based on the phenomenon of fluorescence quenching, which can occur at relatively high dye concentrations. From a concentration of 50 μM on the dye molecules in the measuring solution mutually influence each other. The fluorescence quenching takes place either by formation of dye-dye complexes, which can be excited to fluorescence more poorly or else by shock collision of dye molecules containing, for example, solvent constituents, which lead to the excited molecules releasing their energy to the environment without radiation. Both quenching effects therefore influence the number of emitted photons in comparison to the absorbed photons and thus decrease the fluorescence quantum yield and the fluorescence intensity of the dye.

The NBD compounds C₂ to C₁₂ (12-17) had very similar curves and showed only slight differences in the respective fluorescence intensities. The shortest fatty acid NBD C₂ (12) showed the highest fluorescence intensity of the respective concentrations, while the longest labelled fatty acid NBD C₁₂ (17) had the smallest intensity. It seems that the chain length of the fatty acid also exerted a slight influence on the fluorescence intensity or this deviation could be the result of scattering. For the measurement of fluorescence of the NBD derivatives in combination with HSA—in analogy to the absorption measurement—a concentration of 25 μM was selected that corresponds to a fluorescence intensity in the range of 50 to 80 FI. This range of the fluorescence intensity provides adequate sensitivity.

The concentration curves of the dansyl dyes were approximately linear within concentrations up to 200 μM. Fluorescence quenching was less marked in this concentration range when compared with the NBD compounds. Further concentrations of over 200 μM up to 600 μM were therefore measured. It seems that the fluorescence intensities of the dansyl dyes were also influenced by fluorescence quenching from a certain concentration on. Above 300 μM the hyperbolic course of the curves increased strongly.

### 3.3.2 Dansyl Dyes

The individual curves of the dansyl compounds C₂ to C₁₂ (19-25) differ more strongly from one another than those of the NBD derivatives. As already observed for the absorption measurement of the dansyl derivatives, dansyl C₁₂ (24) again showed the lowest signal intensity of its substance class in the fluorescence measurement. Even the compound dansyl methylamide (25), which bears no acid group, showed only a low fluorescence intensity in comparison to the other derivatives. Thus, there seems to be no correlation between chain length of the respective fatty acid and fluorescence intensity, because the dansyl compound C₂ (19) also provided signals in the lower range of signal intensities. Dansyl C₆ (21) and dansyl C₈ (22) had the highest intensities. A suitable concentration with adequate fluorescence intensity was selected in analogy to the procedure for the NBD derivatives. As for the absorption measurement, a concentration of 100 μM was also suitable for the fluorescence measurements, because at this concentration fluorescence quenching was not seen and this concentration has an adequately large fluorescence intensity in the range from 20 to 60 FI.

The fluorescence intensities of the two dyes used differ markedly. The dansyl dyes showed much lower fluorescence intensities at the same concentrations in comparison to the NBD dyes. Generally, as in the case of the absorption measurement, the dansyl radical is a dye label, which has markedly lower spectroscopic intensities of absorption and emission than the NBD label. However, because for the NBD derivatives fluorescence quenching was available even at low concentrations, contrary to the dansyl derivatives, for which quenching occurred only at higher concentrations, the same maximum fluorescence intensities in the linear measuring range of approximately 150 FI were measured for both dyes.

### 3.3.3 Wave Length Shifts

Characteristic wavelength shifts of fluorescence according to Stoke's law were investigated. For determining the individual emission maxima of the dye concentrations (12-17; 19-25) selected beforehand, emission spectra having an excitation wavelength of 480 nm for NBD and 330 nm for dansyl derivatives were performed. The wavelengths for excitation of the dyes were identical to their absorption wavelengths.

In analogy to the measurement of the absorption spectrum, a higher concentration of the derivatives was also measured, since the emission maxima were found to be more marked. The emission maxima of the other dye derivatives and also the corresponding Stokes shifts are shown in the table below.

TABLE 6
Emission maxima of the dye derivatives with Stokes shifts

| | λₑₘ | λₑₓ | Δλ | | λₑₘ | λₑₓ | Δλ |
|---|---|---|---|---|---|---|---|
| Compound | [nm] | [nm] | [nm] | Compound | [nm] | [nm] | [nm] |
| NBD C₂ | 550 | 476 | 74 | dansyl C₂ | 572 | 326 | 246 |
| (12) | | | | (19) | | | |
| NBD C₄ | 552 | 482 | 70 | dansyl C₄ | 566 | 326 | 240 |
| (13) | | | | (20) | | | |
| NBD C₆ | 556 | 486 | 70 | dansyl C₆ | 562 | 326 | 236 |
| (14) | | | | (21) | | | |
| NBD C₈ | 554 | 486 | 68 | dansyl C₈ | 556 | 326 | 230 |
| (15) | | | | (22) | | | |
| NBD C₁₀ | 556 | 486 | 70 | dansyl C₁₀ | 556 | 326 | 230 |
| (16) | | | | (23) | | | |
| NBD C₁₂ | 556 | 486 | 70 | dansyl C₁₂ | 554 | 322 | 232 |
| (17) | | | | (24) | | | |
| | | | | DMA (25) | 568 | 328 | 240 |
| Mean value | 554 | 484 | 70 | Mean value | 562 | 326 | 236 |

### Example 4

### Influence of HSA on Absorption and Fluorescence of the Dyes

### 4.1 Summary

Using the concentrations and wavelengths selected beforehand further investigations were carried out on HSA. A decreased absorption was shown for NBD derivatives due to HSA binding. No change in absorption was determined for dansyl compounds.

With the exception of the short-chain NBD derivatives C₂ to C₆ (12-14) an increased fluorescence intensity was determined for all dye compounds upon HSA addition. Short-chain NBD derivatives C₂ to C₆ (12-14) seemed not to bind to HSA.

Furthermore, the influence of HSA addition to the dyes on their excitation and emission maxima was examined. The excitation wavelengths of the dyes were not influenced by HSA. The emission maxima showed a shift to shorter wavelengths for almost all of the NBD and dansyl dyes with the exception of the short-chain NBD derivatives C₂ to C₆ (12-14) that did not bind to HSA. The change in fluorescence intensity of the labelled fatty acids upon addition of HSA renders them suitable for competition experiments.

### 4.2 Methods

5.00 ml of a 400 μM solution were prepared from the HSA stock solution. From this solution a geometric dilution series with 7 dilution steps, a dilution factor of 0.5 and a final volume of each concentration of 2.5 ml were prepared.

Defined concentrations of the fluorescence-labelled compounds were selected from the preceding experiment without HSA above and double-concentrated solutions thereof were prepared to take into account the later 1:1 dilution. For each fluorescence-labelled compound (12-17; 19-25) 1.00 ml of these double-concentrations was prepared. 50 μl of the dye solutions were introduced into a 96 well microplate and 50 μl each of the HSA dilution series were added. By mixing the solutions the concentrations of dye derivatives and the HSA solution were halved. The measurements of the absorption and fluorescence were carried out in analogy to the above experimental procedure without HSA.

TABLE 5
Applied and final concentrations of NBD and dansyl compounds.

| | Applied | Final concentration |
|---|---|---|
| | concentration | with HSA |
| NBD derivatives | 50 μM | 25 μM |
| (12-17) | 100 μM | 50 μM |
| Dansyl derivatives | 200 μM | 100 μM |
| (19-25) | 400 μM | 200 μM |

4.3 Absorption of NBD and Dansyl Dyes with HSA

For determining a change in absorption by addition of HSA to the dye solutions (12-17; 19-25), the suitable concentrations for NBD (25 μM) and dansyl derivatives (100 μM) selected beforehand were mixed with increasing concentrations of HSA, in the range from 1.56 to 200 μM, and measured at 480 nm for NBD and 330 nm for dansyl derivatives.

### 4.3.1 NBD Derivatives

For the NBD derivatives (12-17) the absorption intensity decreased for all derivatives with increasing concentration of HSA. By addition of HSA the free NBD derivatives were bound or screened off and therefore absorbed less radiation than before. This effect increased with increasing concentration of HSA.

The compound NBD C₁₂ (17) showed the least absorption power and the signal was also the lowest in combination with HSA. NBD C₄ (13) had the greatest absorption, which was already observed in the experiment without HSA. For the displacement experiments with natural fatty acids and active substances shown below, this indicates that a displacement of a potentially bound NBD fatty acid on HSA had to be accompanied by an increase in absorption.

### 4.3.2 Dansyl Derivatives

Upon addition of HSA to dansyl derivatives—in analogy to the above NBD experiment—no change in the absorption power within the concentration range up to 200 μM for HSA was observed.

For this reason the competition experiments with natural fatty acids shown below were only monitored by fluorescence for the binding experiments of dansyl derivatives to HSA, because there was no absorption signal change in the presence of HSA.

Moreover, the influence of HSA on the respective absorption maxima of the NBD and dansyl compounds (12-17; 19-25) were also investigated. The absorption spectra of all dyes were measured using different concentrations of HSA in the range from 1.56 to 200 μM and compared to the absorption maxima from the previous experiment without HSA (Table 5). The wavelength maxima of the individual dye compounds are shown in Table 6 in the presence and absence of HSA.

TABLE 6
Absorption maxima of the dyes with increasing concentrations of HSA.

| | Absorption | | | Absorption maxima | |
|---|---|---|---|---|---|
| | maxima [nm] | | | [nm] | |
| Compound | without | with | Compound | without | with |
| [25 μM] | HSA | HSA | [100 μM] | HSA | HSA |
| NBD C₂ (12) | 476 | 476 | dansyl C₂ (19) | 326 | 328 |
| NBD C₄ (13) | 482 | 480 | dansyl C₄ (20) | 326 | 328 |
| NBD C₆ (14) | 486 | 482 | dansyl C₆ (21) | 326 | 328 |
| NBD C₈ (15) | 486 | 480 | dansyl C₈ (22) | 326 | 328 |
| NBD C₁₀ (16) | 486 | 478 | dansyl C₁₀ (23) | 326 | 328 |
| NBD C₁₂ (17) | 486 | 476 | dansyl C₁₂ (24) | 322 | 328 |
| | | | DMA (25) | 328 | 326 |
| Mean value | 484 | 479 | Mean value | 326 | 328 |

(The mean value was formed from the determined wavelengths of the increasing HSA concentrations for each dye derivative.)

The NBD dyes showed only a small change in the absorption maximum upon addition of HSA in the average range of 5 nm. This effect, however, was so slightly marked and therefore was neglected. The dansyl dyes behaved similar to the NBD dyes. The average shift for dansyl was 2 nm and was also neglected. The low shifts in the presence of HSA were in the range of the measuring inaccuracy of the microplate reader used.

4.4 Fluorescence of NBD and Dansyl Dyes with HSA

In analogy to the absorption measurement the effect of HSA on the fluorescence of the dyes (12-17; 19-25) was determined. The same concentrations of NBD and dansyl dyes and the same concentration range for HSA as already described above were used.

### 4.4.1 NBD Derivatives

The long-chain NBD compounds C₈ (15), C₁₀ (16) and C₁₂ (17) gave an increase in fluorescence intensity with increasing HSA concentration, whereas the short-chain NBD dyes C₂ (12), C₄ (13) and C₆ (14) produced no changes in fluorescence intensity. This different behavior for the long and short NBD-labelled fatty acids seems to be due to different binding affinities of the NBD derivatives to HSA and indicates that the short-chain NBD dyes NBD C₂ to NBD C₆ (12-14) have weaker or no binding to HSA.

The increase in fluorescence intensity for compounds C₈ to C₁₂ (15-17) upon addition of HSA results from the ‘FRET effect’. The resonance energy transfer takes place between the NBD dyes and the single tryptophan in HSA. An energy transfer occurs if a donor, here tryptophan, releases its energy to an acceptor, here the NBD dye. For this effect to happen, acceptor and donor should be no further apart than 10 nm from one another. The closer donor and acceptor are located, the greater is the energy transfer. An increase in HSA concentration leads to an increased FRET effect for the dyes, because the number of bound fatty acids increases. The hyperbolic curve for NBD C₈ to C₁₂ (15-17) at higher concentrations of HSA indicates a saturation of the effect, i.e. the concentration at which a large proportion of labelled fatty acids is bound to HSA. The shortest fatty acid compound NBD C₂ (12) showed the lowest fluorescence intensity of all derivatives. The dyes NBD C₄ (13) and C₆ (14) had an about identical fluorescence level. For the long-chain derivatives, compound C₈ (15) showed the greatest increase in fluorescence intensity. The increase in fluorescence intensity for NBD derivatives upon binding to HSA was used for competition experiments with natural fatty acids and active substances. A potential displacement of the dye on HSA by competitors results in an increase in free dye in solution and, thus, to a decrease in fluorescence intensity.

### 4.4.2 Dansyl Derivatives

The fluorescence measurement of dansyl derivatives using different concentrations of HSA resulted in similar fluorescence intensity changes as those for the NBD derivatives. In contrast to the NBD dyes, a signal increase in fluorescence intensity due to the FRET effect is seen for all dansyl derivatives. It seems that all dansyl derivatives bind to HSA and that these derivatives have a higher binding affinity to HSA compared to the corresponding NBD dyes. The same as for the HAS-bound NBD derivatives, a hyperbolic saturation curve course was also observed for the dansyl derivatives.

As expected, dansyl methylamide (25), which even without HSA gave only moderate fluorescence intensity, showed the smallest fluorescence increase upon HSA binding. Dansyl C₆ (21), C₈ (22) and C₁₀ (23) compounds showed the highest fluorescence intensities, which corresponds to the experiments without HSA. Like the NBD derivatives C₈ to C₁₂ (15-17) all dansyl derivatives (19-25) are theoretically suitable for competition experiments. Interestingly, there were changes in the emission wavelengths upon addition of different HSA concentrations, especially in the case of dansyl compounds (19-25) (Table 7). To the contrary, NBD dyes showed only slight changes in emission maxima.

TABLE 7
Emission maxima of the dyes with
increasing concentrations of HSA

| Compound | | | | Compound | | | |
|---|---|---|---|---|---|---|---|
| [25 μM] | λₑₘ [nm] | | | [100 μM] | λₑₘ [nm] | | |
| λₑₓ = 480 | without | with | | λₑₓ = 330 | without | with | |
| nm | HSA | HSA | Δλ | nm | HSA | HSA | Δλ |
| NBD C₂ | 550 | 550 | 0 | dansyl C₂ | 572 | 478 | 94 |
| (12) | | | | (19) | | | |
| NBD C₄ | 552 | 550 | 2 | dansyl C₄ | 566 | 478 | 88 |
| (13) | | | | (20) | | | |
| NBD C₆ | 556 | 556 | 0 | dansyl C₆ | 562 | 480 | 82 |
| (14) | | | | (21) | | | |
| NBD C₈ | 554 | 544 | 10 | dansyl C₈ | 556 | 482 | 74 |
| (15) | | | | (22) | | | |
| NBD C₁₀ | 556 | 540 | 16 | dansyl C₁₀ | 556 | 488 | 68 |
| (16) | | | | (23) | | | |
| NBD C₁₂ | 556 | 540 | 16 | dansyl C₁₂ | 554 | 488 | 66 |
| (17) | | | | (24) | | | |
| | | | | DMA (25) | 568 | 488 | 80 |

A further indicator of the extent of the FRET effect is the shift of the emission maxima to smaller wavelengths, which corresponds to a greater energy absorption by the FRET effect. Dyes NBD C₂ to C₆ (12-14) show no FRET effect. A shift of 2 nm is observed for NBD C₄ (13), which however seems due to instrument variation. The long-chain NBD derivatives C₈ to C₁₂ (15-17) indicate a shift in the maxima due to the FRET effect. Shifts for NBD dyes are from 10 to 16 nm in wavelength and are very small compared to those of the dansyl dyes.

For dansyl dyes the C₂ (19) derivative showed the greatest shift of 94 nm to shorter wavelengths and has the greatest energy absorption by the resonance energy transfer. With increasing chain length of the dansyl derivatives this shift effect decreases. In other words, with increasing chain length less energy is transferred to the dansyl derivatives. The longest fatty acid dansyl C₁₂ (24) still showed an about fourfold greater shift, similar to the corresponding NBD compound C₁₂ (17). Dye DMA (25), which has a methyl group instead of a fatty acid, provided for a shift similar to the shift of dansyl C₆ (22). Although the short methyl group is similar to the dansyl C₂-acid (19), its FRET-related energy absorption is lower than for the dansyl C₂ (19). The carboxyl group of the acid seems to play a role as an acceptor for energy absorption. For the NBD derivatives the FRET effect increases with increasing chain length.

In summary, the measurement of fluorescence in the context of the HSA binding has a number of advantages compared to an absorption measurement. For the absorption measurement of NBD derivatives, no difference was seen with regard to the chain length of the dyes.

### Example 5

### Stoichiometry of Labelled Fatty Acids to HSA

### 5.1 Summary

The stoichiometry of NBD- and dansyl-labelled fatty acids bound to HSA was determined by Job plots, also called the “method of continuous variability” as different mole fractions of the respective binding partners are mixed with one another and then measured spectroscopically. Solutions of equimolar concentrations of HSA and dye were mixed such that the mole fractions of the substances were varied but the total molarity of the sample solution stayed constant. For short NBD derivatives C₂ to C₆ (12-14) no binding to HSA was confirmed, whereas binding ratios for long NBD derivatives C₈ to C₁₂ (15-17) to HSA were 1:1. For short dansyl derivatives C₂ to C₈ (19-22) binding ratios for HSA were 2:1, whereas the long dansyl derivatives C₁₀ and C₁₂ (23-24) bound to HSA in the ratio of 4:1. The finding that even dansyl C₂ (19) was bound to HSA led to the question as to what extent the dansyl label itself is recognized by HSA. Because even dansyl methylamide (25) bound to HSA, even though it lacks a carboxyl group, it seems that the dye label could also bind to HSA.

### 5.2 Job Plot of the Fluorescence

For the Job plot the dye and HSA solutions were employed in equimolar amounts. 10.00 ml of 100 μM HSA solution and in each case 2.00 ml of a 100 μM dye solution (12-17; 19-25) were prepared from the above stock solutions. For the Job plot method the dye solutions were mixed with the HSA solution in different ratios as described below in Table 8. The total concentration stayed constant at 100 μM. Only the mole fractions (χ) and the volume fractions (φ) of the different solutions changed. According to the same dilution scheme the dye solutions were also mixed with DPBS buffer for measuring the blank values of the fluorescence-labelled derivatives (see K. C. Ingham, Analytical Biochemistry 68 (1975) 660-663). All solutions were mixed in a 96 well microplate and then pipetted into a 384 well microplate in analogy to the above experiment without HSA. Absorption was not measured. A fluorescence measurement without a wavelength scan was also carried out in analogy to the above experiment without HSA.

TABLE 8
Mole fractions (χ) and volume fractions (φ) of the dye and
HSA solutions used in the Job plot

| nₜₒₜ or Vₜₒₜ | χ or φ | χ or φ | |
|---|---|---|---|
| [μM; μl] | dye | HSA | φ DPBS |
| 100 | 0 | 1.0 | 1.0 |
| 100 | 0.1 | 0.9 | 0.9 |
| 100 | 0.2 | 0.8 | 0.8 |
| 100 | 0.3 | 0.7 | 0.7 |
| 100 | 0.4 | 0.6 | 0.6 |
| 100 | 0.5 | 0.5 | 0.5 |
| 100 | 0.6 | 0.4 | 0.4 |
| 100 | 0.7 | 0.3 | 0.3 |
| 100 | 0.8 | 0.2 | 0.2 |
| 100 | 0.9 | 0.1 | 0.1 |
| 100 | 1.0 | 0 | 0 |

5.2.1 Job Plot of the NBD Compounds with HSA

For NBD derivatives (12-17) and HSA 100 μM solutions were used. The solutions were equimolar. In total 11 solutions per dye derivative were prepared for fluorescence measurement by mixing the two binding partners. Each solution contained different mole fractions of HSA and NBD but total molarity stayed constant. The fluorescence intensities were plotted against the mole fractions of the binding partners. The resulting curve for a binding pair indicates the stoichiometric binding ratio by the position of the maximum value.

The results of the Job plots of the NBD derivatives (12-17) generally gave two different curves. Three of the NBD derivatives gave a curve with a maximum and the remaining derivatives showed a sloping straight line with an increasing mole fraction of HSA.

As mentioned before, a stoichiometric binding ratio of the respective binding partners is indicated by a maximum value. For the long-chain dyes NBD C₈ to NBD C₁₂ (15-17) such a maximum is seen at a ratio of NBD to HSA of approximately 1:1. The longest chain NBD C₁₂ (17) bound to HSA in a ratio of 1:1. The two other derivatives NBD C₈ (15) and C₁₀ (17) gave a maximum value that shifted toward greater mole fractions of HSA. This indicated that the binding ratio of HSA to dye lies at a ratio of about 2:1. This phenomenon seems to be due to poorer HSA binding of the derivatives NBD C₈ (15) and NBD C₁₀ (16) in comparison to compound NBD C₁₂ (17). Because of poorer binding only every second dye is bound. It seems that because of the low amount of HSA, which is insufficient for binding all of the dye, light scattering takes place, which reduces the fluorescence intensity. By adding more HSA, more dye can be bound and fluorescence intensity increases again. When the entire dye is bound at a mole fraction of 0.5 HSA, the intensity of fluorescence decreases by further addition of HSA, because now scattering effects come into play and the fraction of fluorescing dye gets smaller. The binding affinity of the NBD derivatives to HSA increases with increasing chain length of the fatty acid. NBD C₁₂ (17) binds to HSA with the highest affinity.

A second curve, which differed little for compounds NBD C₂ to NBD C₆ (12-14), showed no maximum value. Therefore, no stoichiometric ratio could be determined for these compounds. This confirmed that short-chain NBD derivatives were not bound by HSA and, thus, are not suited for competition experiments. Increasingly adding HSA to short-chain dyes leads to decreasing fluorescence because the HSA has light-scattering effects and the fraction of fluorescent dyes NBD C₂ to NBD C₆ (12-14) decreases.

The determined binding ratios of HSA to NBD were used for competition experiments with natural fatty acids and active substances as described below. For all compounds NBD C₈ to NBD C₁₂ (15-17), the binding ratio was fixed at 1:1.

5.2.2 Job Plot of the Dansyl Compounds with HSA

The Job plot for the dansyl derivatives was carried out in analogy to the NBD dyes. In comparison to the NBD Job plots, it turned out that the dansyl dyes bound to HSA with different stoichiometric binding ratios. Moreover, all dansyl derivatives showed a maximum value in the curve. Consequently, also all short-chain dansyl compounds bind to HSA. Furthermore, dansyl dyes have a higher binding affinity to HSA when compared to corresponding NBD dyes of identical fatty acid chain length.

The curves of the individual dansyl compounds were similar to the curves of the NBD derivatives C₈ to C₁₂ (15-17). Even for a small addition of HSA to the dansyl dyes, scattering effects were produced that decreased fluorescence intensity. Increasing HSA increases fluorescence intensity up to the point where the stoichiometric binding ratio is achieved and all of the dye is bound. Upon further addition of HSA the fluorescent dye in the measuring solution decreases and the scattering effects due to HSA increase, which causes a decrease of the fluorescence intensity. The initial values for zero mole fractions of HSA with dansyl derivatives C₁₀ (23) and C₁₂ (24)—in contrast to the corresponding NBD derivatives—showed a lower fluorescence intensity than for values having a HSA mole fraction of 0.1. This indicates solubility problems for the long-chain dansyl derivatives in the DPBS buffer and is explained by the measured log D_{7.4} measurements. The log D_{7.4} measurement of NBD C₁₂ (17) was in the medium polar range of 2.84, whereas the corresponding dansyl compound C₁₂ (24) showed a log D_{7.4} value of 3.49, which was more strongly lipophilic and thus dissolved more poorly in the aqueous medium of the buffer. The undissolved particles distort the measurements of the fluorescence intensity by light scattering.

The various binding ratios for dansyl derivatives to HSA are summarized in Table 9. For the long-chain dansyl derivatives C₁₀ (23) and C₁₂ (24) an NBD to HSA ratio of 4:1 was determined. All other dansyl derivatives, except for dansyl methylamide (25), bind to HSA in the ratio of 2:1. DMA (25) bound to HSA in the ratio of 1:1. The stoichiometric binding ratio increases with increasing chain length of the labelled dansyl fatty acids.

TABLE 9
Stoichiometric binding ratios of dansyl to HSA (total molarity 100 μM)

| | Binding ratio of |
|---|---|
| Dye | dye to HSA |
| dansyl C₂ (19) | 2:1 |
| dansyl C₄ (20) | 2:1 |
| dansyl C₆ (21) | 2:1 |
| dansyl C₈ (22) | 2:1 |
| dansyl C₁₀ (23) | 4:1 |
| dansyl C₁₂ (24) | 4:1 |
| DMA (25) | 1:1 |

DMA (25), which does not carry a carboxyl group, binds to HSA. Therefore, it seems that for the other dansyl fatty acid derivatives C₂ to C₁₂ (19-24), the binding of HSA is not only mediated via the carboxyl group, but also via the dansyl radical itself. This interfering binding affinity of the dye label, which is overlaid by the carboxyl group binding to HSA, is undesired and will distort competition experiments. Therefore, no competition experiments were carried out with dansyl derivatives (19-25).

### Example 6

### HSA Competition Experiments with NBD-Labelled Fatty Acids

Ligand displacement of fluorescence-labelled fatty acids decreased fluorescence intensity. In competition experiments with C₆ to C₁₆ fatty acids displacement decreased with increasing chain length of the dye. Furthermore, NBD C₁₂ (17) had the greatest binding affinity for HSA; the binding affinity of NBD C₁₀ (16) was minimally smaller and NBD C₈ (15) had the lowest affinity to HSA. This was confirmed by the K_{d} values of the compounds NBD C₁₀ (16) and C₁₂ (17).

### 6.1 Displacement Experiments

For displacement experiments 2.50 ml of a 25 μM HSA solution were used per dye derivative. Due to the subsequent dilution steps the HSA concentration was doubled twice such that 2.50 ml of 100 μl μM solution were prepared.

The dye concentrations (15-17; 23-25) were determined stoichiometrically in the above experiment and are shown in Table 10. The dye concentrations were also doubled twice by the following dilution steps. 2.50 ml of each dye solution were needed. 2.10 ml of the respective dye solution were then mixed with 2.10 ml of the 100 μM HSA solution. The respective concentrations of the solutions halve here once. 50 μl of the mixture per well were introduced into a 96 well microplate. Blank values of the respective concentration of the dye and of the dye-HSA mixture were pipetted onto the same microplate and are shown in Table 10.

TABLE 10
Concentrations of the dye solutions used in the competition experiments

| | Applied | Final | | Blank | Blank |
|---|---|---|---|---|---|
| | dye | dye | | value | value |
| | con- | con- | Dye:HSA | of | of |
| | centration | centration | ratio | dye | HSA & dye |
| NBD C₈ (15) | 100 μM | 25 μM | 1:1 | 25 μM | 25 μM & 25 |
| NBD C₁₀ (16) | | | | | μM |
| NBD C₁₂ (17) | | | | | |
| Dansyl C₁₀ | 400 μM | 100 μM | 4:1 | 100 μM | 25 μM & 100 |
| (23) | | | | | μM |
| Dansyl C₁₂ | | | | | |
| (24) | | | | | |
| Dansyl | 200 μM | 50 μM | 2:1 | 50 μM | 25 μM & 50 |
| methyl-amide | | | | | μM |
| (25) | | | | | |

6.2 Competition with Fatty Acids

The fatty acid microplates were brought to room temperature and 50 μl each of the dilution series were pipetted into the HSA-dye mixture. The concentrations of all solution constituents were thereby halved once again. In analogy to the experiments measuring the fluorescence of the fluorescence-labelled derivatives without HSA above, the solutions were pipetted from the 96 well microplate into a 384 well microplate. Absorption was not measured. The fluorescence measurement without a wavelength scan was carried out in analogy to the above-referenced experiment.

6.2.1 Displacement Experiments of the NBD Derivatives with Natural Fatty Acids on HSA

The competition experiments were carried out with the NBD derivatives C₈ (15), C₁₀ (16) and C₁₂ (17), because these compounds bound to HSA. As competitors, saturated fatty acids, such as caproic acid (C₆), caprylic acid (C₈), capric acid (C₁₀), lauric acid (C₁₂), myristic acid (C₁₄) and palmitic acid (C₁₆) were used here. The non-fluorescent fatty acids selected are bound by HSA. The long-chain fatty acids (C₁₂- C₁₆) show a higher affinity for HSA than the short fatty acids (C₆-C₁₀).

A concentration of 25 μM and a binding ratio of the NBD derivatives to HSA of 1:1 were employed for the displacement studies. For each dye derivative an NBD-HSA mixture with a concentration of 25 μM was prepared. By addition of various concentrations of the saturated fatty acids C₆ to C₁₆ a potential displacement of the respective dyes on HSA was observed.

As already mentioned, an increase in fluorescence intensity was observed upon addition of HSA to the dyes. Due to the displacement by a competitor the fluorescence intensity decreases to the original intensity value that corresponds to the free NBD dye. With an increasing fatty acid concentration the fluorescence intensity decreases to the value at which no NBD C₁₂ (17) is bound to HSA. The longest fatty acid chain length C₁₆ exerts the greatest displacement effect on NBD C₁₂ (17). Furthermore, compound NBD C₁₂ (17) was displaced by the corresponding fatty acid C₁₂. Generally, the displacement effect increased with increasing chain length of the fatty acid. However, fatty acid C₁₀—contrary to fatty acid C₁₂—was no longer capable of displacing bound NBD C₁₂ (17) from HSA. Consequently, the binding affinities of the shorter fatty acids C₆ to C₁₀ to HSA were smaller than those of the dye NBD C₁₂ (17). The binding of the long-chain fatty acids C₁₂ to C₁₆ to HSA was stronger than that of the dye derivative. The displacement experiments with NBD C₈ (15) and NBD C₁₀ (16) were carried out in analogy to the experiment with NBD C₁₂ (17).

The results of the displacement experiment with NBD C₁₀ (16) showed that this derivative was displaced from HSA more easily than NBD C₁₂ (17). NBD C₁₀ was also displaced from HSA by C₁₂ to C₁₆ fatty acids. In analogy to NBD C₁₂ (17) the displacement effect increased with increasing chain length of the fatty acid. Fatty acid C₁₆ also displaced the dye from HSA better than fatty acid C₁₂. Dye NBD C₁₀ (16) was not displaced from HSA by fatty acids C₈ and C₆. It seems that fatty acids C₆ and C₈ have lower binding affinities to HSA than the dye NBD C₁₀ (16). The HSA binding of the long-chain fatty acids C₁₂, C₁₄ and C₁₆ was stronger, whereas the binding of the fatty acid C₁₀ was comparable to the binding of the dye NBD C₁₀ (16).

In the displacement experiment with NBD C₈ (15) the curves were less pronounced than with NBD C₁₂ (17). Nevertheless, compound NBD C₈ (15) was displaced from HSA by fatty acids C₁₆, C₁₄, C₁₂ and C₁₀ as indicated by a decrease of the fluorescence signal. The displacement effect increased with increasing chain length of the fatty acid. Fatty acid C₆ did not displace the dye from HSA. The binding affinities of fatty acids C₁₀ to C₁₆ were greater than those of the dye NBD C₈ (15). Fatty acid C₈ showed a similar binding strength to HSA, whereas fatty acid C₆ was the weaker binding partner in comparison to NBD C₈ (15).

The results of the above displacement experiments for the NBD derivatives C₈ to C₁₂ (15-17) bound to HSA using natural C₆ to C₁₆ fatty acids as competitor ligands are summarized in the following table.

TABLE 11
Summary of the competition experiments with natural fatty acids
C₆ to C₁₆ for the NBD derivatives bound to HSA.

| | | NBD C₁₀ | NBD C₁₂ |
|---|---|---|---|
| | NBD C₈ (15) | (16) | (17) |
| FA C₆ | − | − | − |
| FA C₈ | unclear | − | − |
| FA C₁₀ | + | unclear | − |
| FA C₁₂ | + | + | + |
| FA C₁₄ | + | + | + |
| FA C₁₆ | + | + | + |

(+ stands for displacement; − for no displacement)

Generally, the binding of NBD dyes to HSA depends on their chain length. Of the three derivatives tested dye NBD C₁₂ (17) bounds best to HSA. NBD C₈ had the lowest affinity for HSA and NBD C₁₀ showed a binding strength which lies between these two.

6.3 Competition with Ibuprofen and Warfarin

400 μl of a 2000 μM ibuprofen and warfarin solution were employed per dye derivative. These 400 μl were diluted geometrically with a dilution factor of 0.5 in 7 dilution steps such that a residual volume of 200 μl of each concentration remained. 50 μl each of the dilution series were pipetted into the HSA-dye mixture. The concentrations of all constituents were thereby halved again.

In analogy to the experiments measuring the fluorescence of the fluorescence-labelled derivatives without HSA above, the solutions were pipetted from the 96 well microplate into a 384 well microplate. Absorption was not measured in this experiment. The fluorescence measurement without a wavelength scan was carried out in analogy to the above-referenced experiment.

For investigation of the binding pocket of the dye NBD C₁₂ (17) on HSA, a further displacement experiment was carried out using the competitors ibuprofen and warfarin. Warfarin binds to Sudlow's site I and ibuprofen to Sudlow's site II. For displacement, an NBD-HSA mixture with a concentration of 25 μM was prepared in analogy to the displacement experiment using the fatty acids as described above. The two competitors were then added to this mixture in different concentrations and the fluorescence intensities were measured. If the dye was displaced by its competitors, the binding site for dye NBD C₁₂ (17) on HSA would be established as a binding pocket for the active substance.

Both resulting competition curves showed a signal increase in the fluorescence intensity. Therefore, NBD C₁₂ (17) was not displaced on HSA by the active substance competitors. Consequently, the binding site of the dye was not identified as binding site for the two competitors. Compound NBD C₁₂ (17) neither bound the Sudlow's site I nor the Sudlow's site II, but to one of the other seven fatty acid binding pockets on HSA.

### 6.4 Determination of the K_{d} Constant

Two geometric dilution series were prepared from the dye stock solutions of NBD C₁₀ (16) and of NBD C₁₂ (17). The first dilution with a dilution factor of 0.5 starting from a 200 μM solution comprised 6 dilution steps and a residual volume of 250 μl per well. Likewise, the second geometric series with the dilution factor of 0.5 consisted only of one dilution step and was prepared from a 300 μM solution with a residual volume of 250 μl per well. Also, 2.00 ml of a 50 μM solution of the HSA stock solution were prepared. 50 μl of the respective concentrations of NBD C₁₀ (16) or NBD C₁₂ (17) were introduced into a 96 well microplate and 50 μl of the HSA solution were pipetted into each well. All concentrations in the wells were halved by mixing. Moreover, the blank value without HSA was measured for each NBD concentration. A fluorescence measurement without a wavelength scan was carried out in analogy to the above-referenced experiment.

### 6.5 FRET Interactions

For determining FRET (Förster resonance energy transfer) interactions of NBD C₁₂ (17) with amino acid Trp214 of HSA, concentrations of a dye-HSA mixture according to Table 8 were applied in an amount of 250 μl for each case.

TABLE 12
Concentrations of the dye-HSA mixtures for FRET experiments

| | HSA | |
|---|---|---|
| Mixture no. | fraction | Dye fraction (17; 24) |
| 1 | 25 μM | — |
| 2 | 25 μM | 5 μM |
| 3 | 25 μM | 10 μM |
| 4 | 25 μM | 15 μM |
| 5 | 25 μM | 20 μM |
| 6 | 25 μM | 25 μM |
| 7 | — | 25 μM |

100 μl of mixture 7 were pipetted into a 96 well microplate and a wavelength scan for the absorption in the range from 250 nm to 700 nm was carried out. In each case 15 μl of the residual solutions 1 to 6 were pipetted four times into a black 384 well microplate and emission spectra of the solution in the range from 270 nm to 700 nm were recorded at 290 nm excitation wavelength of the Trp.

For dye NBD C₁₂ (17) the FRET effect between the tryptophan of the HSA and the dye itself was investigated more precisely. The emission spectrum of the amino acid tryptophan overlapped with the absorption spectrum of the NBD dye, because the energy of the donor to be transferred must lie in the region of the possible energy absorption of the acceptor. This is the case when the two spectra overlap. For measurement, both solutions were employed in identical concentrations (25 μM) and the units of the fluorescence intensities (FIᵣₑₗ) and the absorption strength (ODᵣₑₗ) were standardized. An excitation wavelength of 290 nm was utilized for the emission spectrum of tryptophan.

In the next step, different concentrations of NBD C₁₂ (17) were added to the HSA solution until a ratio of NBD to HSA of 1:1 was reached, and the emission spectra were measured. The emission maximum for tryptophan was at a wavelength of 330 nm with a fluorescence intensity in the range of 170 FI. Subsequent addition of NBD dye C₁₂ (17) caused a decrease in the tryptophan emission intensity to 80 FI and an increase in the NBD C₁₂ (17) fluorescence to 15 FI.

The efficiency of the energy transfer occurring was calculated using the equation 3.4.

$$E = \frac{{R}_{0}^{6}}{{R}_{0}^{6} + {r}^{6}} = 1 - \frac{I}{{I}_{0}} \qquad 3.4$$

The measured fluorescence intensity I of the donor tryptophan and the acceptor NBD C₁₂ (17) was 82 FI and the intensity I₀ of the donor tryptophan without dye was 171 FI. An energy efficiency of approximately 52% was calculated.

The distance R₀, at which a 50% energy exchange takes place, was calculated according to the following equation. Furthermore, by transformation of said equation the real distance r of donor and acceptor was determined.

R₀=9.78·10³(κ²·n⁻⁴·φ_{D}·J)^{1/6}

For NBD derivatives calculated J overlapping intervals with tryptophan were in the order of magnitude of 5-9×10⁻¹⁵ M⁻¹·κ² is indicated in the literature with a value of 0.67 and the refractive index n for DPBS buffer (0.01 M, pH 7.4) is 1.333. The fluorescence quantum yield for tryptophan is 0.15.

By means of these parameters the distance R₀ for NBD C₁₂ (17) and tryptophan was estimated to be in a range of 23 to 25 Å. For the real radius r a distance from 22 to 24 Å results, which corresponds to 2.2 to 2.4 nm. This value indicates how close the acceptor NBD C₁₂ (17) and the donor tryptophan in HSA come for eliciting FRET effect.

1. A composition comprising:
a fluorescent-labelled fatty acid; and
a fatty acid binding compound;
wherein
the fatty acid component of the fluorescent-labelled fatty acid binds the fatty acid binding compound; and
the fluorescent component of the fluorescent-labelled fatty acid and the fatty acid binding compound interact to elicit FRET (Förster resonance energy transfer) effects.

2. The composition according to claim 1, wherein the fluorescent component of the fluorescent-labelled fatty acid is selected from the group consisting of 4-nitrobenzo-2-oxa-1,3-diazole (NBD) and 5-(dimethylamino)naphthalene-1-sulfonyl (dansyl).

3. The composition according to claim 1, wherein the fatty acid component of the fluorescent-labelled fatty acid is selected from the group consisting of linear or branched, saturated or unsaturated fatty acids.

4. The composition according to claim 1, wherein the fatty acid binding compound is selected from the group consisting of proteins comprising at least one tryptophan.

5. The composition according to claim 1, wherein
the fluorescent component is 4-nitrobenzo-2-oxa-1,3-dizole (NBD);
the fatty acid component is selected from the group consisting of linear and saturated fatty acids having 8 to 25 carbon atoms; and
the fatty acid binding compound is albumin.

6. The composition according to claim 5, wherein the concentration of the fluorescent-labelled fatty acid is 2 μM to 50 μM.

7. A method for identifying and/or characterizing a compound of interest, comprising the steps of:
providing a fluorescent-labelled fatty acid;
providing a fatty acid binding compound that binds to the fatty acid and interacts with the fatty acid to elicit FRET (Förster resonance energy transfer) effects;
contacting the fluorescent-labelled fatty acid with the fatty acid binding compound under conditions that allow for (a) the binding of the fatty acid to the fatty acid binding compound and for (b) FRET (Förster resonance energy transfer) effects;
contacting the fluorescent-labelled fatty acid bound to the fatty acid binding compound with a compound of interest under conditions that allow for (a) the binding of the fatty acid to the fatty acid binding compound and for (b) FRET (Förster resonance energy transfer) effects;
determining the change in fluorescence; and
optionally calculating the binding affinity of the compound of interest to the fatty acid binding compound.

8. The method according to claim 7, wherein the fluorescent component of the fluorescent-labelled fatty acid is selected from the group consisting of 4-nitrobenzo-2-oxa-1,3-diazole (NBD) and 5-(dimethylamino)naphthalene-1-sulfonyl (dansyl).

9. The method according to claim 7, wherein the fatty acid component of the fluorescent-labelled fatty acid is selected from the group consisting of linear and saturated fatty acids having 2 to 25 carbon atoms.

10. The method according to claim 7, wherein the fatty acid binding compound is selected from the group consisting of proteins comprising at least one tryptophan.

11. The method according to any of claim 7, wherein
the fluorescent component is 4-nitrobenzo-2-oxa-1,3-dizole (NBD);
the fatty acid component is selected from the group consisting of linear and saturated fatty acids having 8 to 25 carbon atoms; and
the fatty acid binding compound is albumin.

12. A kit comprising
a fluorescent-labelled fatty acid; and
a fatty acid binding compound;
wherein
the fatty acid component of the fluorescent-labelled fatty acid binds the fatty acid binding compound; and
the fluorescent component of the fluorescent-labelled fatty acid and the fatty acid binding compound interact to elicit FRET (Förster resonance energy transfer) effects.

13. (canceled)

14. A method of manufacturing a composition according to claim 1, the method comprising admixing the fluorescent-labelled fatty acid and the fatty acid binding compound under conditions allowing for a binding of the fatty acid component of the fluorescent-labelled fatty acid and the fatty acid binding compound.

15. A method of manufacturing a kit according to claim 12, the method comprising assembling the different components of the kit to form a spatial and/or functional unit.

16. The method of claim 7, wherein the fluorescent-labelled fatty acid and the fatty acid binding compound are provided as a composition comprising:
a fluorescent-labelled fatty acid; and
a fatty acid binding compound;
wherein
the fatty acid component of the fluorescent-labelled fatty acid binds the fatty acid binding compound; and
the fluorescent component of the fluorescent-labelled fatty acid and the fatty acid binding compound interact to elicit FRET (Förster resonance energy transfer) effects.

17. The method of claim 7, wherein the fluorescent-labelled fatty acid and the fatty acid binding compound are provided as a kit comprising:
a fluorescent-labelled fatty acid; and
a fatty acid binding compound;
wherein
the fatty acid component of the fluorescent-labelled fatty acid binds the fatty acid binding compound; and
the fluorescent component of the fluorescent-labelled fatty acid and the fatty acid binding compound interact to elicit FRET (Förster resonance energy transfer) effects.
